(19) United States
(12) Patent Application Publication
(10) Pub. No.: US 2022/0380809 A1
(43) Pub. Date: Dec. 1, 2022

(54) DELIVERY OF INTACT CRISPR/CAS9 PROTEIN USING SUPRAMOLECULAR NANOPARTICLE (SMNP) VECTORS

(71) Applicant: The Regents of the University of California, Oakland, CA (US)

(72) Inventors: Hsian-Rong Tseng, Oakland, CA (US); Qian Ban, Oakland, CA (US); Yazhen Zhu, Oakland, CA (US); Peng Yang, Oakland, CA (US)

(73) Assignee: The Regents of the University of California, Oakland, CA (US)

(21) Appl. No.: 17/775,159

(22) PCT Filed: Nov. 13, 2020
(86) PCT No.: PCT/US2020/060533

§ 371 Date: May 6, 2022

Related U.S. Application Data
(60) Provisional application No. 62/935,905, filed on Nov. 15, 2019.

(51) Int. Cl. C12N 15/88; C12N 9/22; C12N 15/11; C12N 15/90

(52) U.S. Cl. CPC C12N 15/88; C12N 9/22; C12N 15/11; C12N 15/907; C12N 2800/80; C12N 2310/20

(57) ABSTRACT

Compositions, systems and methods for delivering CRISPR/Cas9-based genome editing system to a cell.

### CROSS-REFERENCE OF RELATED APPLICATION

This application claims priority to U.S. Provisional Application No. 62/935,905 filed Nov. 15, 2019; the entire contents of all of which are hereby incorporated by reference.

### BACKGROUND

### 1. Technical Field

The field of the currently claimed embodiments of this invention relates to compositions, systems and methods for delivering CRISPR/Cas9-based genome editing system to a cell.

### 2. Discussion of Related Art

Since its proof-of-concept demonstration¹, the CRISPR/Cas9-based genome editing system² has generated a lot of excitement in the scientific community because it is faster, cheaper, more accurate, and more efficient than other existing genome editing methods.

Compared to the viral transfection approaches, co-delivery of Cas9 protein along with a single guide RNA (sgRNA) offers a straightforward strategy for genome editing without the concerns associated with viral integration. Although a variety of approaches for co-delivery of Cas9 protein and sgRNA have been reported³⁻⁴, it remains a challenge to further improve the co-delivery efficiency.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a composition for delivering an endonuclease to a cell, the composition including: a plurality of self-assembled supramolecular nanoparticles (SMNPs), each of the plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; a plurality of terminating components, each having a single terminating binding element that binds to remaining binding regions of one of the plurality of binding components by forming a second inclusion complex; the endonuclease; and a nucleotide sequence including a recognition sequence specific to the endonuclease. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle. In such an embodiment, the endonuclease and the nucleotide sequence form an endonuclease and nucleotide sequence complex, and the endonuclease and nucleotide sequence complex are encapsulated within each of the plurality of self-assembled supramolecular nanoparticles (SMNPs).

An embodiment of the invention relates to a system for delivering an endonuclease to a cell, the system including: a plurality of self-assembled supramolecular nanoparticles (SMNPs), each of the plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; a plurality of terminating components, each having a single terminating binding element that binds to remaining binding regions of one of the plurality of binding components by forming a second inclusion complex; the endonuclease; and a nucleotide sequence having a recognition sequence specific to the endonuclease; and a device for capturing the cell, the device including: a substrate; and a plurality of nanowires at least one of attached to or integral with a surface of the substrate such that each nanowire of the plurality of nanowires has an unattached end. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the endonuclease and the nucleotide sequence form an endonuclease and nucleotide sequence complex, the endonuclease and nucleotide sequence complex is encapsulated within each of the plurality of self-assembled supramolecular nanoparticles (SMNPs), and the plurality of self-assembled supramolecular nanoparticles (SMNPs) are reversibly attached to the plurality of nanowires.

An embodiment of the invention relates to a method for delivering an endonuclease to a cell including: providing a plurality of self-assembled supramolecular nanoparticles (SMNPs); and contacting the cell with at least one of the plurality of self-assembled supramolecular nanoparticles (SMNPs) such that the at least one of the plurality of self-assembled supramolecular nanoparticles (SMNPs) is taken up by the cell. In such an embodiment, the plurality of self-assembled supramolecular nanoparticles (SMNPs) include: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; a plurality of terminating components, each having a single terminating binding element that binds to remaining binding regions of one of the plurality of binding components by forming a second inclusion complex; the endonuclease; and a nucleotide sequence including a recognition sequence specific to the endonuclease. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the endonuclease and the nucleotide sequence form an endonuclease and nucleotide sequence complex, and the endonuclease and nucleotide sequence complex is encapsulated within the plurality of self-assembled supramolecular nanoparticles (SMNPs).

### BRIEF DESCRIPTION OF THE DRAWINGS

Further objectives and advantages will become apparent from a consideration of the description, drawings, and examples.

FIG. 1 is a schematic representations of the self-assembled approach for preparation of Cas9·sgRNA-encapsulated supramolecular nanoparticles (Cas9·sgRNA⊂SMNPs), according to an embodiment of the invention. Three types of molecular recognition mechanisms, including (i) specific binding between Cas9 protein and sgRNA for formation of an anionic Cas9·sgRNA complex, (ii) the Ad/CD-based molecular recognition for generation of SMNP vectors with cationic PEI/PAMAM hydrogel cores, and (iii) electrostatic interactions that facilitate incorporation of anionic Cas9·sgRNA into SMNPs, were harnessed for the self-assembly of Cas9·sgRNA⊂SMNPs by simply mixing Cas9·sgRNA with the 3 SMNP molecular building blocks, i.e., CD-PEI, Ad-PAMAM, and Ad-PEG.

FIG. 2 is a schematic illustration of the unique mechanism governing a “supramolecular nanosubstrate-mediated delivery (SNSMD)” strategy for both in vivo and in vitro settings, according to an embodiment of the invention. The multivalent molecular recognition between the Ad motifs on Adamantane-Grafted Silicon Nanowire Substrates (Ad-SiNWS) and the β-cyclodextrin (CD) motifs on the surfaces of SMNPs leads to dynamic assembly and local enrichment of Cas9·sgRNA⊂SMNPs onto Ad-SiNWS. As GFP-expressing cells (i.e., U87-GFP) settle onto Ad-SiNWS, the intimate contact between the cell membrane and the nanowires on Ad-SiNWS facilitate the uptake of Cas9·sgRNA⊂SMNPs into the cells, resulting in highly efficient genome editing to silent GFP expression in U87-GFP cells.

FIG. 3 is an SEM image of a U87 cell on Ad-SiNWS, on which Cas9·sgRNA⊂SMNPs (100-150 nm in diameters) were grafted via supramolecular assembly, according to an embodiment of the invention. As U87-GFP cells settle onto the substrates, the intimate contact between the cell membrane and the nanowires on Ad-SiNWS facilitate the uptake of Cas9·sgRNA⊂SMNPs into the cells.

FIGS. 4A and 4B are a panel of images and a graph showing genome editing to silence GFP expression of U87-GFP cells via the treatment of Cas9·sgRNA⊂SMNPs (40 μg mL⁻¹), according to an embodiment of the invention. FIG. 4A is a panel of fluorescence microscopy images and FIG. 4B is a graph of quantitative image cytometry data of U87-GFP cells 0, 24, 36, 48, 60, and 72 hr after Cas9·sgRNA⊂SMNPs treatment.

FIGS. 5A and 5B are a panel of images and a graph showing genome editing to silence GFP expression of U87-GFP cells at different doses of Cas9·sgRNA⊂SMNPs (5 to 40 μg mL⁻¹), according to an embodiment of the invention. FIG. 5A is a panel of fluorescence microscopy images and FIG. 5B is a graph of fluorescence signals of U87-GFP cells measured 48 hr after Cas9·sgRNA⊂SMNPs treatment.

FIGS. 6A-6C are schematic illustrations of the mechanism governing a combined SMNP/SNSMD strategy for CRISPR/Cas9-mediated GFP gene disruption by introducing Cas9·sgRNA-GFP⊂SMNPs into GFP-U87 cells, according to an embodiment of the invention.

FIGS. 7A-7F are illustrations, data graphs and fluorescent images showing a combined SMNP/SNSMD strategy for delivering an EGFP-Cas9·sgRNA complex into U87 cells, according to an embodiment of the invention.

FIGS. 8A-8F are scanning electron microscopy (SEM) images and transmission electron microscopy (TEM) images of 8%-TAT-grafted EGFP-Ca9·sgRNA⊂SMNPs, according to an embodiment of the invention.

FIGS. 9A-9G are illustrations, fluorescent images, and data graphs showing CRISPR/Cas9-mediated GFP gene disruption in GFP-U87 cells using a combined SMNP/SNSMD strategy, according to an embodiment of the invention.

FIGS. 10A-10D are fluorescent images and data graphs showing results of two sequential treatments of Cas9·sgRNA-GFP⊂SMNPs to GFP-U87 cells via the combined SMNP/SNSMD strategy, according to an embodiment of the invention.

FIGS. 11A-11D are schematics and data graphs showing CRISPR/Cas9-mediated deletion of exons 45-55 of dystrophin gene in AC16 cells using a combined SMNP/SNSMD strategy, according to an embodiment of the invention.

FIG. 12 shows the DNA sequence for exons 44-55 for deletion in the dystrophin gene, according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some aspects of the invention include supramolecular nanoparticles (SMNPs), having a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; and a plurality of terminating components, each having a single terminating binding element that binds to remaining binding regions of one of the plurality of binding components by forming a second inclusion complex. SMNPs are described in in U.S. Pat. No. 9,845,237 and U.S. Patent Application No. 20160000918, each of which is herein incorporated in its entirety by reference. The plurality of binding components, plurality of cores, and the plurality of terminating components self-assemble when brought into contact to form the supramolecular magnetic nanoparticle (SMNP).

The plurality of binding components, plurality of cores, and the plurality of terminating components bind to each other by one or more intermolecular forces. Examples of intermolecular forces include hydrophobic interactions, biomolecular interactions hydrogen bonding interactions, π-π interactions, electrostatic interactions, dipole-dipole interactions, or van der Waals forces. Examples of bimolecular interactions include DNA hybridization, a protein-small molecule interaction (e.g. protein-substrate interaction (e.g. a streptavidin-biotin interaction) or protein-inhibitor interaction), an antibody-antigen interaction or a protein-protein interaction. Examples of other interactions include inclusion complexes or inclusion compounds, e.g. adamantane-β-cyclodextrin complexes or diazobenzene-α-cyclodextrin complexes. Generally, the intermolecular forces binding the components of the SMNP structure are not covalent bonds.

Some embodiments of the invention include a device for capturing a cell. Examples of such devices are described in U.S. Pat. No. 9,140,697, which is hereby incorporated by references in its entirety. A further non-limiting example of such a device is a Silicon Nanowire Substrates (SiNWS). In embodiments of the invention, the device includes a substrate; and a plurality of nanowires at least one of attached to or integral with a surface of the substrate such that each nanowire of the plurality of nanowires has an unattached end. In some embodiments, the plurality of nanowires are configured to reversibly attach to self-assembled supramolecular nanoparticles (SMNPs).

In some embodiment the device for capturing a cell includes a substrate having a nanostructured surface region. Also, in some embodiments, a plurality of binding agents are attached to the nanostructured surface region of the substrate. However, binding agents are not required for the device to bind to target cells. The nanostructured surface region includes a plurality of nanostructures, each having a longitudinal dimension and a lateral dimension. As a sample is placed on the device, biological cells are selectively captured by the binding agents and the plurality of nanostructures acting in cooperation (in embodiments having binding agents). When present, the binding agent or agents employed will depend on the type of biological cell(s) being targeted. Conventional binding agents are suitable for use in some of the embodiments of the present invention. Non-limiting examples of binding agents include antibodies, nucleic acids, oligo- or polypeptides, cellular receptors, ligands, aptamers, biotin, avidin. Coordination complexes, synthetic polymers, and carbohydrates. In some embodiments of the present invention, binding agents are attached to the nanostructured surface region using conventional methods. The method employed will depend on the binding agents and the material used to construct the device. Non-limiting examples of attachment methods include non-specific adsorption to the surface, either of the binding agents or a compound to which the agent is attached or chemical binding, e.g., through self-assembled monolayers or silane chemistry. In some embodiments, the nanostructured surface region is coated with streptavidin and the binding agents are biotinylated, which facilitates attachment to the nanostructured surface region via interactions with the streptavidin molecules.

In some embodiments of the present invention, the nanostructures increase the surface area of the substrate and increase the probability that a given cell will come into contact. In these embodiments, the nanostructures can enhance binding of the target cells by interacting with cellular surface components such as microvilli, lamellipodia, filopodia, and lipid-raft molecular groups. In some embodiments, the nanostructures have a longitudinal dimension that is equal to its lateral dimension, wherein both the lateral dimension and the longitudinal dimension is less than 1 mm, i.e., nanoscale in size. In other embodiments, the nanostructures have a longitudinal dimension that is at least ten times greater than its lateral dimension. In further embodiments, the nanostructures have a longitudinal dimension that is at least twenty times greater, fifty times greater, or 100 times greater than its lateral dimension. In some embodiments, the lateral dimension is less than 1 mm. In other embodiments, the lateral dimension is between 1-500 nm. In further embodiments, the lateral dimension is between 30-400 nm. In still further embodiments, the lateral dimension is between 50-250 nm. In some embodiments, the longitudinal dimension is at least 1 mm long. In other embodiments, the longitudinal dimension is between 1-50 mm long. In other embodiments, the longitudinal dimension is 1-25 mm long. In further embodiments, the longitudinal dimension is 5-10 mm long. In still further embodiments, the longitudinal dimension is at least 6 mm long. The shape of the nanostructure is not critical. In some embodiments of the present invention, the nanostructure is a sphere or a bead. In other embodiments, the nanostructure is a strand, a wire, or a tube. In further embodiments, a plurality of nanostructure contains one or more of nanowires, nanofibers, nanotubes, nano-pillars, nanospheres, or nanoparticles.

Embodiments of the invention are related to compositions, systems, and/or methods for delivering CRISPR/Cas9-based genome editing system to a cell. In some embodiments, a self-assembled nano-particle is configured to encapsulate and deliver a functional Cas9 enzyme and a guide RNA to a cell for editing of a genomic DNA sequence (including, but not limited to a gene, and intron, and/or and exon). In some embodiments, a self-assembled nano-particle is configured to encapsulate and deliver a nucleic acid sequence encoding for a Cas9 enzyme. In some embodiments, a self-assembled nano-particle is configured to encapsulate and deliver a protein or peptide; non-limiting examples of such a protein or peptide include a recombinant protein or peptide, or a replacement protein or peptide. In some embodiments, a self-assembled nano-particle is configured to encapsulate and deliver a nucleotide sequence encoding a protein or a peptide.

Some embodiments of the invention are related to methods for genome editing in a cell. In some such embodiments, a target cell is contacted with a self-assembled nano-particle configured to encapsulate and deliver a functional Cas9 enzyme and a guide RNA to the cell for editing of a target genomic DNA sequence. In some embodiments, the self-assembled nano-particle is configured to encapsulate and deliver a nucleic acid sequence encoding for a Cas9 enzyme. In some embodiments, the target cell is contacted with two different self-assembled nano-particles: a first self-assembled nano-particle configured to encapsulate and deliver to the cell a functional Cas9 enzyme and a guide RNA, or a nucleic acid sequence encoding for a Cas9 enzyme; and a second self-assembled nano-particle configured to encapsulate and deliver a protein or peptide or a nucleic acid sequence encoding a protein or peptide.

An embodiment of the invention relates to a composition for delivering an endonuclease to a cell having: a plurality of self-assembled supramolecular nanoparticles (SMNPs), each of the plurality of self-assembled supramolecular nanoparticles (SMNPs) having: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; a plurality of terminating components, each having a single terminating binding element that binds to remaining binding regions of one of the plurality of binding components by forming a second inclusion complex; the endonuclease; and a nucleotide sequence including a recognition sequence specific to the endonuclease. The plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the endonuclease and the nucleotide sequence form an endonuclease and nucleotide sequence complex, and the endonuclease and nucleotide sequence complex are encapsulated within each of the plurality of self-assembled supramolecular nanoparticles (SMNPs).

An embodiment of the invention relates to the composition above, where the endonuclease is a CRISPR associated protein 9 (Cas9), and where the nucleotide sequence is a single guide RNA (sgRNA).

An embodiment of the invention relates to the composition above, where each of the plurality of self-assembled supramolecular nanoparticles (SMNPs) has a diameter of between 40 nanometers and 600 nanometers.

An embodiment of the invention relates to the composition above, where the plurality of binding components includes polythylenimine, poly(L-lysine), or poly(β-amino ester).

An embodiment of the invention relates to the composition above, where the plurality of binding regions includes beta-cyclodextrin, alpha-cyclodextrin, gamma-cyclodextrin, cucurbituril or calixarene.

An embodiment of the invention relates to the composition above, where the plurality of cores includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the composition above, where the at least one core binding element includes adamantane, azobenzene, ferrocene or anthracene.

An embodiment of the invention relates to the composition above, where the plurality of terminating components includes polyethylene glycol (PEG) or poly(propylene glycol) (PGG).

An embodiment of the invention relates to the composition above, where the single terminating binding element includes adamantane, azobenzene, ferrocene or anthracene.

An embodiment of the invention relates to a system for delivering an endonuclease to a cell including: a plurality of self-assembled supramolecular nanoparticles (SMNPs), each of the plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; a plurality of terminating components, each having a single terminating binding element that binds to remaining binding regions of one of the plurality of binding components by forming a second inclusion complex; the endonuclease; and a nucleotide sequence including a recognition sequence specific to the endonuclease; and a device for capturing the cell, the device including: a substrate; and a plurality of nanowires at least one of attached to or integral with a surface of the substrate such that each nanowire of the plurality of nanowires has an unattached end. In such embodiments, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the endonuclease and the nucleotide sequence form an endonuclease and nucleotide sequence complex, the endonuclease and nucleotide sequence complex is encapsulated within each of the plurality of self-assembled supramolecular nanoparticles (SMNPs), and the plurality of self-assembled supramolecular nanoparticles (SMNPs) are reversibly attached to the plurality of nanowires.

An embodiment of the invention relates to the system above, where the endonuclease is a CRISPR associated protein 9 (Cas9), and where the nucleotide sequence is a single guide RNA (sgRNA).

An embodiment of the invention relates to the system above, where each of the plurality of self-assembled supramolecular nanoparticles (SMNPs) has a diameter of between 100 nanometers and 150 nanometers.

An embodiment of the invention relates to the system above, where the plurality of binding components includes polythylenimine, poly(L-lysine), or poly(β-amino ester).

An embodiment of the invention relates to the system above, where the plurality of binding regions includes beta-cyclodextrin, alpha-cyclodextrin, gamma-cyclodextrin, cucurbituril or calixarene.

An embodiment of the invention relates to the system above, where the plurality of cores includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the system above, where the at least one core binding element includes adamantane, azobenzene, ferrocene or anthracene.

An embodiment of the invention relates to the system above, where the plurality of terminating components includes polyethylene glycol or poly(propylene glycol) (PGG).

An embodiment of the invention relates to the system above, where the single terminating binding element includes adamantane azobenzene, ferrocene or anthracene.

An embodiment of the invention relates to the system above, where the plurality of nanowires are grafted with adamantane, adamantane azobenzene, ferrocene, or anthracene.

An embodiment of the invention relates to the system above, where the plurality of nanowires has a diameter of between 40 nanometers and 600 nanometers.

An embodiment of the invention relates to the system above, where the plurality of nanowires includes silicon, gold, silver, SiO2, or TiO2.

An embodiment of the invention relates to a method for delivering an endonuclease to a cell including: providing a plurality of self-assembled supramolecular nanoparticles (SMNPs); and contacting the cell with at least one of the plurality of self-assembled supramolecular nanoparticles (SMNPs) such that the at least one of the plurality of self-assembled supramolecular nanoparticles (SMNPs) is taken up by the cell. In such an embodiment, the plurality of self-assembled supramolecular nanoparticles (SMNPs) include: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; a plurality of terminating components, each having a single terminating binding element that binds to remaining binding regions of one of the plurality of binding components by forming a second inclusion complex; the endonuclease; and a nucleotide sequence including a recognition sequence specific to the endonuclease. In such an embodiment, the plurality of binding components and the plurality of cores self-assemble when brought into contact to form the plurality of self-assembled supramolecular nanoparticles (SMNPs), the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle, the endonuclease and the nucleotide sequence form an endonuclease and nucleotide sequence complex, and the endonuclease and nucleotide sequence complex is encapsulated within the plurality of self-assembled supramolecular nanoparticles (SMNPs).

An embodiment of the invention relates to the method above, where the endonuclease is a CRISPR associated protein 9 (Cas9), and where the nucleotide sequence is a single guide RNA (sgRNA).

An embodiment of the invention relates to the method above, where each of the plurality of self-assembled supramolecular nanoparticles (SMNPs) has a diameter of between 100 nanometers and 150 nanometers.

An embodiment of the invention relates to the method above, where the plurality of binding components includes polythylenimine poly(L-lysine) or poly(β-amino ester).

An embodiment of the invention relates to the method above, where the plurality of binding regions includes beta-cyclodextrin, alpha-cyclodextrin, gamma-cyclodextrin, cucurbituril and calixarene.

An embodiment of the invention relates to the method above, where the plurality of cores includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the method above, where the at least one core binding element includes adamantane azobenzene, ferrocene or anthracene.

An embodiment of the invention relates to the method above, where the plurality of terminating components includes polyethylene glycol (PEG) or poly(propylene glycol) (PGG).

An embodiment of the invention relates to the method above, where the single terminating binding element includes adamantane azobenzene, ferrocene or anthracene.

An embodiment of the invention relates to the method above, where the plurality of self-assembled supramolecular nanoparticles (SMNPs) are reversibly attached to a plurality of nanowires, and where the plurality of nanowires are at least one of attached to or integral with a surface of a substrate such that each nanowire of the plurality of nanowires has an unattached end.

An embodiment of the invention relates to the method above, where the plurality of nanowires are grafted with adamantane azobenzene, ferrocene or anthracene.

An embodiment of the invention relates to the method above, where the plurality of nanowires has a diameter of between 40 nanometers and 600 nanometers.

An embodiment of the invention relates to the method above, where the plurality of nanowires includes silicon, gold, silver, SiO2 or TiO2.

### EXAMPLES

### Example 1

Previously, a convenient, flexible, and modular self-assembly approach for the preparation of supramolecular nanoparticles (SMNPs) from a small collection of molecular building blocks through a multivalent molecular recognition based on adamantane (Ad) and β-cyclodextrin (CD) motifs was demonstrated⁸; methods for assembling such particles are described in U.S. Pat. No. 9,845,237 and U.S. Patent Application No. 20160000918, which are each herein incorporated in their entirety by reference. Such a self-assembly synthetic strategy enables arbitrary control of the sizes, surfaces chemistry, zeta potentials and payloads of the resulting SMNPs. Such control opens up many interesting opportunities for biomedical applications, including positron emission tomography (PET) imaging⁸, magnetic resonance imaging (MRI)⁹, photothermal treatment of cancer cells¹⁰, highly efficient gene delivery¹¹, and on-demand delivery of a chemo therapy drug. Furthermore, the use of SMNP vectors for delivering intact (unmodified) transcription factors (TFs) with superior efficiency was also explored. An objective was to achieve the encapsulation of TFs into cationic SMNP-vectors by introducing anionic characteristics to the TF. A DNA plasmid with a matching recognition sequence specific to a TF can be employed to form an anionic TFDNA complex, which can be subsequently encapsulated into SMNPs, resulting in TF-encapsulated SMNPs (TF·DNA⊂SMNPs).

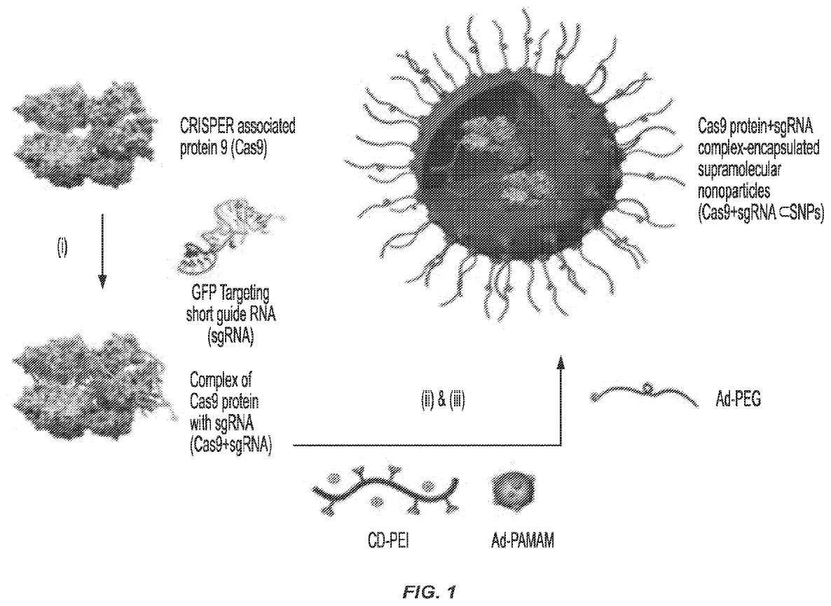

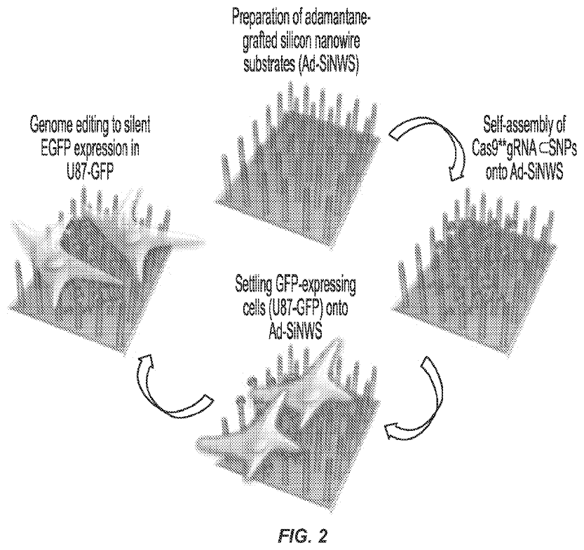

Herein, the utility of SMNP vectors for co-delivery of Cas9 protein and sgRNA is shown. Using a self-assembled synthetic approach, the feasibility (FIG. 1) to encapsulate Cas9 protein and sgRNA complex (Cas9·sgRNA) into SMNP vectors is shown. To further enhance the intracellular delivery efficiency of Cas9·sgRNA-encapsulated SMNPs (Cas9·sgRNA⊂SMNPs), the “supramolecular nanosubstrate-mediated delivery (SNSMD)” strategy¹² (FIG. 2) was adopted. Adamantane-grafted silicon nanowire substrates (Ad-SiNWS) were prepared according to previously published procedures. Upon treating Ad-SiNWS with a PBS solution containing Cas9·sgRNA⊂SMNPs, the multivalent molecular recognition between the Ad motifs on Ad-SiNWS and the β-cyclodextrin (CD) motifs on Cas9·sgRNA⊂SMNPs leads to dynamic assembly and local enrichment of Cas9·sgRNA⊂SMNPs onto Ad-SiNWS. As cells settle onto the substrates, the intimate contact between the cell membrane and the nanowires on Ad-SiNWS facilitate the uptake of Cas9·sgRNA⊂SMNPs into the cells, resulting in highly efficient delivery of Cas9·sgRNA for genome editing.

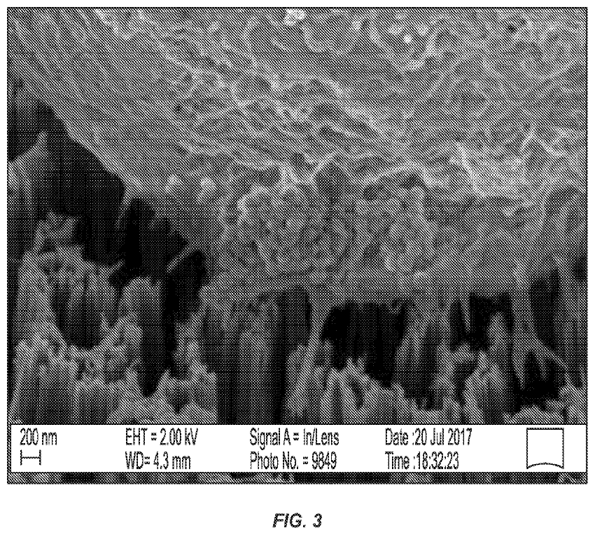

The sizes of Cas9·sgRNA⊂SMNPs were characterized by dynamic light scattering and scanning electron microscopy (SEM). The results suggest that the sizes of Cas9·sgRNA⊂SMNPs between 100 and 150 nm are adjustable. The self-assembly of Cas9·sgRNA⊂SMNPs onto Ad-SiNWS can also be characterized (visualized) by SEM (FIG. 3), supporting the working mechanism of SNSMD strategy described in an earlier publication¹².

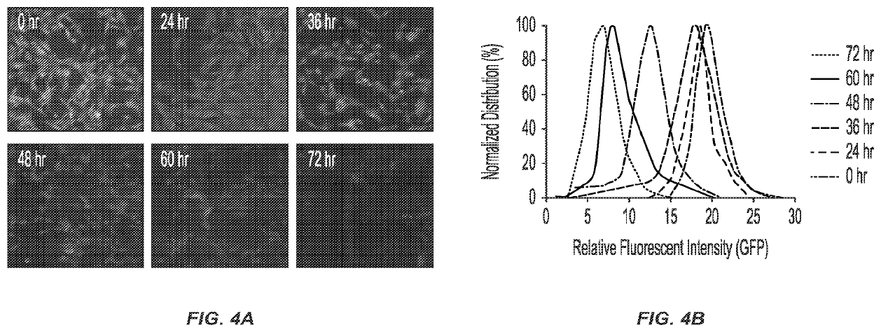

To test the efficacy of Cas9·sgRNA⊂SMNPs for genome editing, a GFP-expressing U87 cell line (U87-GFP) as a model system was utilized. Here, GFP-targeting sgRNA and Cas9 protein were co-encapsulated into SMNP vectors using the self-assembly approach described above to give Cas9·sgRNA⊂SMNPs. Using “supramolecular nanosubstrate-mediated delivery (SNSMD)” strategy¹² (FIG. 2), Cas9·sgRNA⊂SMNPs were grafted onto Ad-SiNWS. As U87-GFP cells settle onto the substrates, the intimate contact between the cell membrane and the nanowires on Ad-SiNWS facilitate the uptake (FIG. 3) of Cas9·sgRNA⊂SMNPs into the cells, resulting in silencing of GFP signals as the result of targeted DNA cleavage and repair by the endogenous NHEJ pathway. The time dependent fluorescence microscopy images and quantitative image cytometry data of Cas9·sgRNA⊂SMNPs-treated U87-GFP cells are summarized in FIGS. 4A and 4B. More than 95% of Cas9·sgRNA⊂SMNPs-treated U87-GFP cells reduced their fluorescence signals as the result of successful genome editing. The GFP expression of the U87-GFP cells started to decline 24-36 h post Cas9·sgRNA⊂SMNPs-treatment and reached background levels 72 h post treatment.

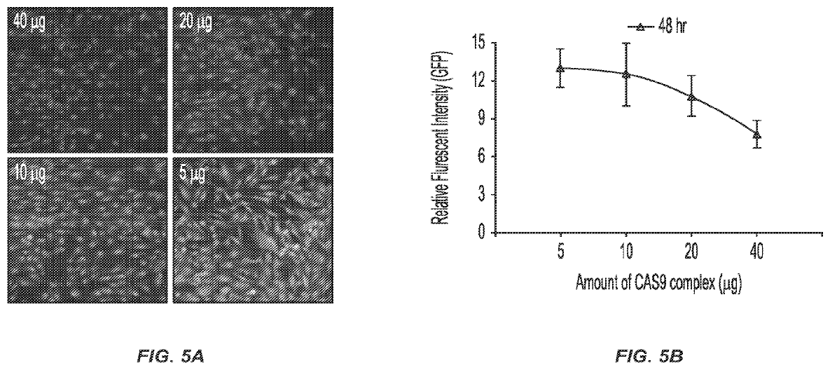

Dose-dependent genome editing studies (FIGS. 5A and 5B) were conducted by treating U87-GFP cells with Cas9·sgRNA⊂SMNPs at different dosages (5, 10, 20, and 40 μg mL⁻¹), suggesting an optimal treatment dosage of 40 μg mL⁻¹.

In conclusion, the results demonstrate the feasibility of applying “supramolecular nanosubstrate-mediated delivery (SNSMD)” strategy to co-deliver Cas9 and sgRNA into targeted cells in a highly efficient manner. The uniqueness of the self-assembly synthetic strategy for the preparation of Cas9·sgRNA⊂SMNPs has to do with the combined use of three types of molecular recognition mechanisms, including: (i) specific binding between Cas9 protein and matching sgRNA for formation of an anionic Cas9·sgRNA complex, (ii) the Ad/CD-based molecular recognition for generation of SMNP vectors with cationic hydrogel cores, and (iii) electrostatic interactions that facilitate encapsulation of anionic Cas9·sgRNA into SMNP vectors. The “supramolecular nanosubstrate-mediated delivery (SNSMD)” strategy¹² (FIG. 2) allows for dynamic assembly and local enrichment of Cas9·sgRNA⊂SMNPs from the surrounding solution/medium onto Ad-SiNWS. As cells settle onto the substrates, the intimate contact between the cell membrane and the nanowires on Ad-SiNWS facilitate the uptake of Cas9·sgRNA⊂SMNPs into the cells, resulting in highly efficient delivery of Cas9·sgRNA for genome editing.

### REFERENCES FROM BACKGROUND AND EXAMPLE 1

1 Jinek, M. et al. A programmable dual-RNA-guided DNA endonuclease in adaptive bacterial immunity. Science 337, 816-821, doi:10.1126/science.1225829 (2012).
2 Hsu, P. D., Lander, E. S. & Zhang, F. Development and applications of CRISPR-Cas9 for genome engineering. Cell 157, 1262-1278, doi:10.1016/j.cell.2014.05.010 (2014).
3 Zuris, J. A. et al. Cationic lipid-mediated delivery of proteins enables efficient protein-based genome editing in vitro and in vivo. Nat Biotechnol 33, 73-80, doi:10.1038/nbt.3081 (2015).
4 Sun, W. et al. Self-assembled DNA nanoclews for the efficient delivery of CRISPR-Cas9 for genome editing. Angew Chem Int Ed Engl 54, 12029-12033, doi:10.1002/anie.201506030 (2015).
5 Wang, M. et al. Efficient delivery of genome-editing proteins using bioreducible lipid nanoparticles. Proc Natl Acad Sci USA 113, 2868-2873, doi:10.1073/pnas.1520244113 (2016).
6 Mout, R. et al. Direct Cytosolic Delivery of CRISPR/Cas9-Ribonucleoprotein for Efficient Gene Editing. ACS Nano 11, 2452-2458, doi:10.1021/acsnano.6b07600 (2017).
7 Ha, J. S. et al. Poly-sgRNA/siRNA ribonucleoprotein nanoparticles for targeted gene disruption. J Control Release 250, 27-35, doi: 10.1016/j.jconrel.2017.02.007 (2017).
8 Wang, H. et al. A supramolecular approach for preparation of size-controlled nanoparticles. Angew Chem Int Ed Engl 48, 4344-4348, doi:10.1002/anie.200900063 (2009).
9 Chen, K.-J. et al. A small MRI contrast agent library of gadolinium(III)-encapsulated supramolecular nanoparticles for improved relaxivity and sensitivity. Biomaterials In Press, DOI: 10.1016/j.biomaterials.2010.11.043, doi:10.1016/j.biomaterials.2010.11.043.
10 Wang, S. et al. Photothermal effects of supramolecularly assembled gold nanoparticles for the targeted treatment of cancer cells. Angew Chem Int Ed Engl 49, 3777-3781, doi:10.1002/anie.201000062 (2010).
11 Wang, H. et al. A rapid pathway toward a superb gene delivery system: programming structural and functional diversity into a supramolecular nanoparticle library. ACS Nano 4, 6235-6243, doi:10.1021/nn101908e (2010).
12 Peng, J. et al. Molecular recognition enables nanosubstrate-mediated delivery of gene-encapsulated nanoparticles with high efficiency. ACS Nano 8, 4621-4629, doi:10.1021/nn5003024 (2014).

### Example 2

CRISPR/Cas9 represents an efficient, precise and cost-effective genomic editing technology, offering a versatile therapeutic solution for a wide range of human diseases. Current CRISPR/Cas9 delivery relies on virus vectors, which suffer from the limitation of packaging capacity and safety concerns. A non-viral delivery method is much needed to fully realize the therapeutic potential of CRISPR/Cas9. Herein, it is shown that the Cas9 protein and sgRNA can be encapsulated into supramolecular nanoparticle (SMNP) vectors (Cas9·sgRNA⊂SMNPs) and then co-delivered into target cell nuclei via the combined SMNP/SNSMD strategy. Utilizing a U87 glioblastoma cell line as a model system, a variety of parameters such as the size of SMNP, the SMNP vector and payload ratio and the time course of Cas9 proteins entering target cells were examined. Further, the dose-dependent and time-dependent CRISPR/Cas9-mediated gene disruption in a GFP-expressing U87 cell line (GFP-U87) was examined. An optimized formulation was shown to be highly efficient in disrupting GFP expression. Finally, the utility of such optimized formulation in co-delivering Cas9 protein and two sgRNAs in human cardiomyocytes cell line (AC16), enabling highly efficient deletion of exons 45-55 (up to 708 kb) of the dystrophin gene, which mutations lead to Duchenne muscular dystrophy (DMD), a severe genetic muscle disease was shown. These results could facilitate the development of novel gene therapy approaches.

The clustered regularly interspaced short palindromic repeats, CRISPR-associated protein 9 (CRISPR/Cas9) system is shifting its role from an RNA-guided genetic adaptive immune system in prokaryotes to a rapidly developing site-specific gene editing method.¹⁻³ The CRISPR/Cas9 gene editing system is composed of two crucial components, i.e., the Cas9 endonuclease, and an engineered short, single-guide RNA (sgRNA), which form a ribonucleoprotein complex, Cas9·sgRNA.⁴ Based on a simple base-pairing mechanism, Cas9·sgRNA recognizes and cuts the target DNA site, precisely introducing a double-strand break (DSB) on the gene.⁵⁻⁶ Following the formation of a DSB, endogenous DNA repair can occur via a non-homologous end joining (NHEJ) pathway,⁷ which serves as the foundation of CRISPR/Cas9-mediated gene disruption.⁸⁻¹⁰ A pair of sgRNAs have been used to generate a large gene deletion, offering a more general therapeutic solution for some monogenic diseases such as Duchenne muscular dystrophy (DMD)¹¹⁻¹⁴ and Leber congenital amaurosis type 10 (LCA10)¹⁵.

Although the CRISPR/Cas9 technology has showed considerable therapeutic potential in a wide range of diseases, a large body of challenges still lie ahead for its translation into therapies, such as off-target, delivery and editing efficiency.¹⁶ Among them, gene delivery presents a key challenge for robust implementation of CRISPR/Cas9 gene editing both in vitro and in vivo.¹⁷⁻¹⁸ So far, the majority of CRISPR/Cas9 delivery relies on viral vectors, such as: lentivirus (LV),¹⁹ adenovirus (AV),²⁰ and adeno-associated virus (AAV)²¹⁻²²due to their practical merits such as easy construction, good production titer, and high transgene expression.¹⁷ However, limitations of packaging capacity (<4.7 kb)²³ and safety concerns of insertional mutagenesis, inflammatory responses, and immunogenicity associated with viral delivery still linger. Alternative solutions have been developed to explore non-viral vectors,^{18, 24} including lipids,²⁵⁻²⁶ polymers²⁷⁻²⁹ and nanoparticles³⁰⁻³³ for delivery of CRISPR/Cas9 system. The CRISPR/Cas9 system can be introduced in three forms: DNA,^{27, 31, 34} mRNA,³⁵⁻³⁸ and protein³⁹⁻⁴³. Compared to non-viral deliveries of Cas9 DNA and Cas9 mRNA, direct delivery of Cas9·sgRNA represents the most straightforward strategy. In principle, non-viral delivery of Cas9·sgRNA has two major advantages: i) rapid gene editing approach, as it skips gene transcription and/or translation; and ii) transient gene editing with consequent reduced off-target effects and toxicity. Due to the large size of Cas9 protein (˜160 kDa), there is a need for more effective delivery vectors.¹⁸ Previously, a convenient and flexible self-assembled synthetic approach for producing supramolecular nanoparticle⁴⁴ (SMNP) vectors by mixing three common molecular building blocks, i.e., (3-cyclodextrin(CD)-grafted branched polyethyleneimine (CD-PEI), adamantane-grafted polyamidoamine dendrimer (Ad-PAMAM), and Ad-grafted poly(ethylene glycol) (Ad-PEG) was demonstrated. The interplay of multivalent molecular recognition between adamantane (Ad) and β-cyclodextrin (CD) motifs⁴⁴ allows modular control over the sizes, surface chemistry, and payloads of SMNP vectors, with a diversity of imaging⁴⁴⁻⁴⁵ and therapeutic applications.⁴⁶⁻⁴⁸ To further improve the delivery efficiency of SMNP vectors, a substrate-mediated delivery strategy⁴⁹, a.k.a., supramolecular nanosubstrate-mediated delivery (SNSMD) was developed, by which Ad-grafted silicon nanowire substrates (Ad-SiNWS) were employed to facilitate the uptake of SMNP vectors into the cells. Here, the multivalent Ad/CD molecular recognition drives dynamic assembly and local enrichment of SMNPs onto Ad-SiNWS. Once the cells settle onto the substrates, the intimate contact between the cell membrane and the nanowires led to highly efficient delivery of SMNP vectors. Moreover, it is feasible to carry out multiple rounds of SMNP delivery on the same batch of cells (by sequential additions of SMNP) without regenerating/reloading the substrates after each single-use.⁵⁰ The combined use of SMNP vector and SNSMD (i.e., a combined SMNP/SNSMD strategy) offers a powerful solution for delivery of Cas9·sgRNA for highly efficient gene editing in cells.

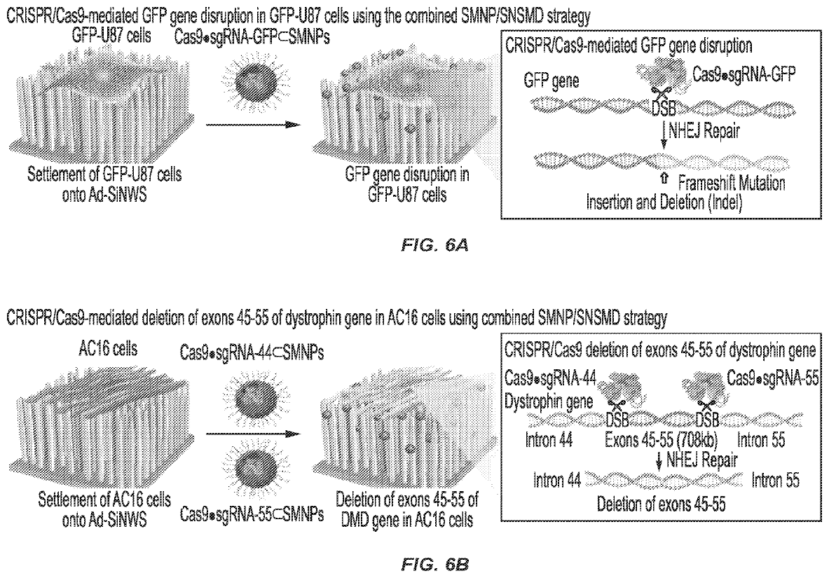

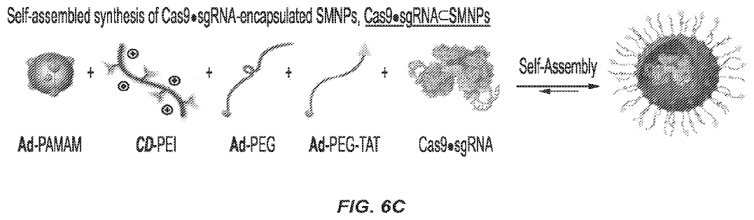

Here, the combined SMNP/SNSMD strategy facilitated the delivery of Cas9·sgRNA into cells settled on Ad-SiNWS for CRISPR/Cas9-mediated gene disruption (FIG. 6A) and deletion (FIG. 6B). The Cas9·sgRNA was encapsulated into SMNP vectors to form Cas9·sgRNA⊂SMNPs via a self-assembled synthetic approach (FIG. 6C). In search of an optimal formulation of Cas9·sgRNA⊂SMNPs, EGFP-labeled Cas9 protein was employed in a quantitative fluorescent imaging study, where U87 glioblastoma cell line was used as a model system. By conducting small-scale combinatorial screening, an optimal formulation of TAT-grafted EGFP-Cas9·sgRNA⊂SMNPs (average size=120±20 nm and 8% TAT coverage) that gave highest cell uptake was identified and subjected to time-dependent imaging studies using U87 cells. Single-cell image analysis unveiled that a maximum EGFP-Cas9 accumulation in the nuclei of majority cells (92%) could be achieved 3 h post EGFP-Ca9·sgRNA⊂SMNPs treatment. Such an optimal formulation was then employed to co-deliver Cas9 protein and GFP-targeting sgRNA (Cas9·sgRNA-GFP) into GFP-expressing U87 cell line (GFP-U87). The Cas9·sgRNA-GFP could disrupt GFP gene as a result of in frameshift mutation, which was induced by CRISPR/Cas9-mediated DSB, followed by DNA repair via NHEJ pathway (FIG. 6A). Both fluorescence microscopy and T7 endonuclease I (T7E1) assay were employed to quantify/monitor the reduction of GFP signals and to test the frequency of insertions and deletions (indels) at the target GFP locus, respectively. Finally, the combined SMNP/SNSMD strategy was used to edit dystrophin gene mutations in an in vitro cell model, human cardiomyocytes cell line (AC16 cells). As mutations of the dystrophin gene directly cause DMD, which is a severe inherited devastating muscle disease, approximately affecting 1 out of 5000 newborn males.⁵³ It causes a wide range of physical consequences, including cardiac associated disease, eventually leading to the death, since it is reported that cardiomyopathies in DMD patients (which are present in ˜90% of DMD patients) are emerging as a main cause of morbidity and mortality.⁵⁴ In addition, many therapies aiming at skeletal muscle treatment failed to improve cardiac function.⁵⁵ On the other hand, approximately 60% of mutations causing DMD occur within exons 45-55 of the dystrophin gene.⁵⁶ It has been demonstrated that CRISPR/Cas9-mediated deletion of exons 45-55 could produce an internally deleted dystrophin protein, resulting in rescued disease phenotype.¹¹⁻¹³ Here, CRISPR/Cas9-mediated deletion of exons 45-55 (up to 708 kb) of dystrophin gene in human AC16 cells (FIG. 6C) is demonstrated, by co-delivering a pair of Cas9·sgRNA complexes (i.e., Cas9·sgRNA-44 targeting introns 44 and Cas9·sgRNA-55 targeting introns 55), which were separately encapsulated in SMNP vectors (i.e., Cas9·sgRNA-44⊂SMNPs and Cas9·sgRNA-55⊂SMNPs, respectively). After deletion of exons 45-55 of dystrophin gene, the 3′ end of intron 44 and the 5′ end of intron 55 joined together via NHEJ pathway. T7E1 assay, polymerase chain reaction (PCR) and Sanger sequencing were employed to conform the precise deletion of exons 45-55 of dystrophin gene in AC16 cells.

FIGS. 6A-6C are schematic illustrations of the mechanism governing a combined SMNP/SNSMD strategy for CRISPR/Cas9-mediated GFP gene disruption by introducing Cas9·sgRNA-GFP⊂SMNPs into GFP-U87 cells, according to an embodiment of the invention. FIG. 6A shows Cas9·sgRNA-GFP could disrupt GFP gene as a result of in frameshift mutation, which is induced by CRISPR/Cas9-mediated DSB, followed by DNA repair via NHEJ pathway. FIG. 6B shows the combined SMNP/SNSMD strategy for CRISPR/Cas9-mediated deletion of exons of 45-55 of dystrophin gene by delivering Cas9·sgRNA-44⊂SMNPs and Cas9·sgRNA-55⊂SMNPs into human AC16 cells. After deletion of exons 45-55 of dystrophin gene, the 3′ end of intron 44 and the 5′ end of intron 55 joined together via NHEJ pathway. FIG. 6C shows a self-assembled synthetic approach adopted for preparation of Cas9·sgRNA⊂SMNPs through ratiometric mixing of four SMNP molecular building blocks (i.e., CD-PEI, Ad-PAMAM, Ad-PEG, and Ad-PEG-TAT), and Cas9·sgRNA complex.

Results and Discussion

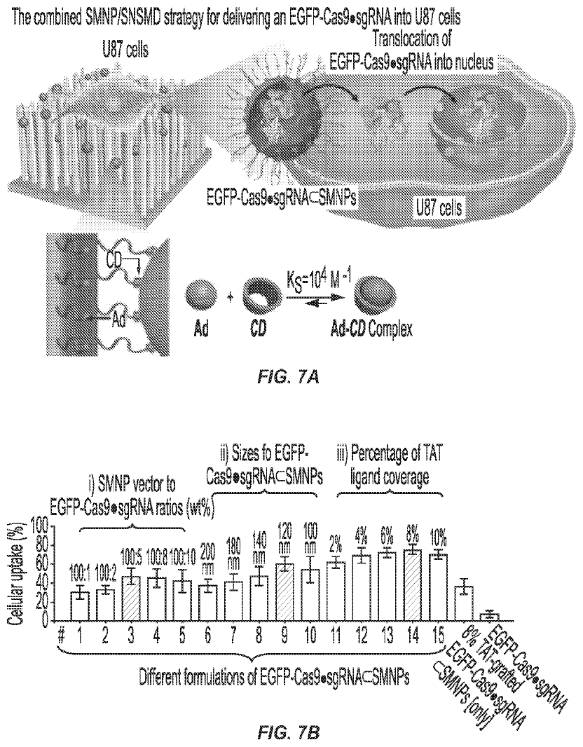

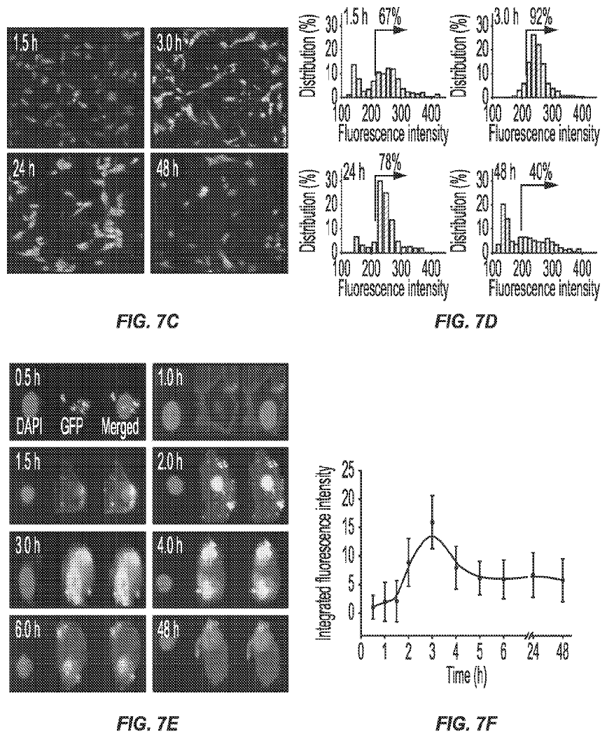

In previous studies^{[29]} SMNP vectors were utilized for co-encapsulating a transcription factor (TF) and a DNA plasmid was used to prepare Cas9·sgRNA⊂SMNPs through stoichiometric mixing of Cas9·sgRNA and four SMNP molecular building blocks (FIG. 6C). Based on the combined SMNP/SNSMD strategy, a three-step optimization was adopted in search of Cas9·sgRNA⊂SMNP formulations that give optimal cell-uptake performance. To allow for quantitative imaging of Cas9 protein by fluorescence microscopy, EGFP-labeled Cas9 protein (EGFP-Cas9, GenCrispr, New Jersey) was used in the uptake study of U87 cells (FIG. 7A). Three batches of EGFP-Cas9·sgRNA⊂SMNPs (FIG. 7B) were formulated via systemically modulating i) the SMNP/EGFP-Cas9·sgRNA weight ratios (100:1, 100:3, 100:5, 100:8, and 100:10); ii) SMNP sizes (100-200 nm); and iii) the coverage of a membrane penetration ligand, TAT (2-10%).^{[30]} The resulting EGFP-Ca9·sgRNA⊂SMNPs and TAT-EGFP-Cas9·sgRNA⊂SMNPs were first subjected to dynamic light scattering (DLS) analysis to characterize their hydrodynamic sizes. For cell-uptake studies, each formulation of EGFP-Cas9·sgRNA⊂SMNPs (containing 1.0 μg of EGFP-Cas9) was added to a well (in a 24-well plate), in which an Ad-SiNWS (1×1 cm²) was immersed with 1.0 mL of Dulbecco's modified Eagle's medium (DMEM). As the result of supramolecular assembly, EGFP-Cas9·sgRNA⊂SMNPs were quickly enriched and grafted onto Ad-SiMWS from the medium. Prior to settling the cells onto Ad-SiMWS, U87 cells were starved in serum-free DMEM overnight (10 h) to synchronize cells to G0/G1 phases of cell cycle.⁵⁸ Thereafter, 1×10⁵ U87 cells were introduced into each well. Co-delivery efficiency of EGFP-Cas9·sgRNA into U87 cells was quantified by fluorescence microscopy 24 h after treatment. First, it was examined how the weight ratios (wt %) between SMNP vectors and EGFP-Cas9·sgRNA affect cell uptake. The results (FIG. 7B) suggest the higher cell uptake (47%) was achieved at the ratio (wt %) of 100:5. Using this formulation ratio, next was studied how the sizes of Cas9·sgRNA⊂SMNPs affect cell uptake. By altering the mixing ratio of Ad-PAMAM and CD-PEI in each formulation, a second batch of EGFP-Ca9·sgRNA⊂SMNPs with five different sizes was prepared, ranging from 100 nm to 200 nm. EGFP-Cas9·sgRNA⊂SMNPs with 120-nm size showed the best cell uptake (60%). Based on this synthetic formula, TAT-grafted EGFP-Cas9·sgRNA⊂SMNPs with TAT coverage ranging between 2% to 10% were prepared. These studies revealed that EGFP-Ca9·sgRNA⊂SMNPs with 8% TAT coverage exhibited an optimal delivery performance up of 75%. Thus, the optimal synthetic formulation that gave 120-nm 8%-TAT-grafted EGFP-Cas9·sgRNA⊂SMNPs was identified, and this formulation was subjected to time-dependent imaging studies. Three control studies, i.e., U87 cells treated with 8%-TAT-grafted EGFP-Cas9·sgRNA⊂SMNPs (no Ad-SiNWS), U87 cells treated with EGFP-Cas9·sgRNA complex (without SMNP vectors) and U87 cells treated with PBS solution were conducted in parallel, highlighting the crucial role of both functional components (Ad-SiNWS and SMNP vectors) of the combined SMNP/SNSMD strategy.

It is crucial to note that, after the cell uptake and dynamic disassembly of EGFP-Cas9·sgRNA⊂SMNPs, EGFP-Cas9 protein is expected to translocate into cell nuclei where the gene editing happens. To characterize translocation of EGFP-Cas9 into to cell nuclei using the combined SMNP/SNSMD strategy, a time-dependent quantitative imaging study was conducted on individual U87 cells under the optimal delivery condition. Multiple cell uptake studies were performed in parallel and terminated at 0.5, 1.0, 1.5, 2.0, 3.0, 4.0, 6.0, and 48 h after treatment. After DAPI nuclear staining, the U87 cells on Ad-SiNWS were subjected to high-resolution microscopy imaging (Nikon Ti2), followed by single-cell image analysis to quantify EGFP-Cas9 signals on the footprints of individual cells. FIG. 7C compiles serial fluorescent micrographs of the U87 cells at 1.5, 3.0, 24, and 48 h after the treatment with EGFP-Cas9·sgRNA⊂SMNPs via the combined SMNP/SNSMD strategy. FIG. 7C shows the histograms of single-cell EGFP-Cas9 uptake at the respective times, suggesting highest cell uptake (92% of U87 cells) was observed at 3.0 h. Thereafter, slow decay of EGFP-Cas9 accumulation was observed. In parallel, the accumulation EGFP-Cas9 in cell nuclei was also analyzed. FIG. 7E compiles serial fluorescent micrographs of individual U87 cells at 0.5, 1.0, 1.5, 2.0, 3.0, 4.0, 6.0, and 48 h post treatment. FIG. 7F shows time-dependent EGFP-Cas9 accumulation in the U87 cells' nuclei, unveiling maximum nuclear accumulation of EGFP-Cas9 at 3.0 h.

FIGS. 7A-7F are illustrations, data graphs and fluorescent images showing a combined SMNP/SNSMD strategy for delivering an EGFP-Cas9·sgRNA complex into U87 cells, according to an embodiment of the invention. Specifically, FIG. 7A shows the combined SMNP/SNSMD strategy for delivering an EGFP-Cas9·sgRNA complex into U87 cells. FIG. 7B shows three batches of EGFP-Cas9·sgRNA⊂SMNPs were formulated via systemically modulating i) the weight ratios (wt %) between SMNP vector and EGFP-Cas9·sgRNA payload, ii) sizes of EGFP-Cas9·sgRNA⊂SMNPs, and iii) the percentages of TAT ligand coverage, followed by cellular uptake studies. For comparison purpose, we kept the EGFP-Cas9 concentration identical (1.0 μg per well) in all studies. An optimal formulation that gave 120-nm 8%-TA-grafted EGFP-Cas9·sgRNA⊂SMNPs (*) was identified. FIG. 7C shows serial fluorescent micrographs of the U87 cells taken at 1.5, 3, 24, and 48 h after their settlement onto the Ad-SiNWs loaded with 120-nm 8%-TAT-grafted EGFP-Cas9·sgRNA⊂SMNPs. FIG. 7D shows graphs demonstrating that by performing single-cell image analysis to quantify EGFP-Cas9 signals in the micrographs shown in FIG. 7C, histograms of single-cell EGFP-Cas9 uptake were obtained for the respective times. The optimal cell uptake (92% of U87 cells) was observed at 3 h post cell settlement. FIG. 7E shows serial fluorescence micrographs of individual U87 cells depict dynamic accumulation of EGFP-Cas9 signals in cell nuclei at 0.5, 1.0, 1.5, 2, 3, 4, 6, and 48 h post cell settlement. FIG. 7F is a graph showing time-dependent EGFP-Cas9 accumulation in cell nuclei, unveiling maximum nuclear accumulation of EGFP-Cas9 at 3.0 h.

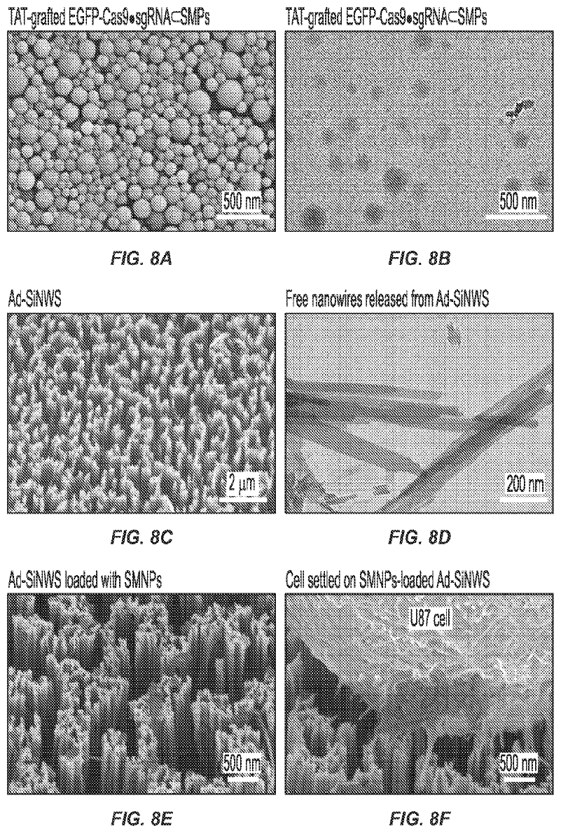

In addition to DLS measurement, scanning electron microscope (SEM) imaging and transmission electron microscopy (TEM) were utilized to characterize 8%-TAT-grafted EGFP-Cas9·sgRNA⊂SMNPs which exhibit an optimal EGFP-Cas9 delivery performance. The SEM and TEM micrographs showed these nanoparticles had homogeneous spherical morphology with size of 110±45 nm (FIGS. 8A and 8B), which was similar with the hydrodynamic size (120±15 nm) obtained by DLS. The surface-charge densities of Cas9·sgRNA⊂SMNPs were determined by zeta potential measurements in PBS buffer solution, which suggest that the 8%-TAT-grafted Cas9·sgRNA⊂SMNPs carry zeta potentials of +20±5 mV. The SEM and TEM images of the Ad-SiNWS showed that the diameters and lengths of Ad-SiNWS are ca. 30-80 nm and 5-10 μm, respectively (FIGS. 8C and 8D). The self-assembly of EGFP-Cas9·sgRNA⊂SMNPs onto Ad-SiNWS (FIG. 8E) and the interactions between cells and Ad-SiNWS (FIG. 8F) can also be visualized by SEM, supporting the working mechanism of the SMNP/SNSMD strategy described in earlier studies.⁵¹

FIGS. 8A-8F are scanning electron microscopy (SEM) images and transmission electron microscopy (TEM) images of 8%-TAT-grafted EGFP-Cas9·sgRNA⊂SMNPs, according to an embodiment of the invention. Specifically, FIG. 8A is a scanning electron microscopy (SEM) image. FIG. 8B is a transmission electron microscopy (TEM) image. FIG. 8C is a SEM image Ad-grafted silicon nanowire substrates (Ad-SiNWS), which were prepared from wet-etching followed by covalent functionalization of Ad motifs. FIG. 8D is a TEM image of free nanowires released from Ad-SiNWS. FIG. 8E is an image showing an SEM image upon exposure of EGFP-Cas9·sgRNA⊂SMNPs in the medium to Ad-SiNWS. FIG. 8F shows a U87 cell settled on Ad-SiNWS loaded with EGFP-Cas9·sgRNA⊂SMNPs as visualized by SEM.

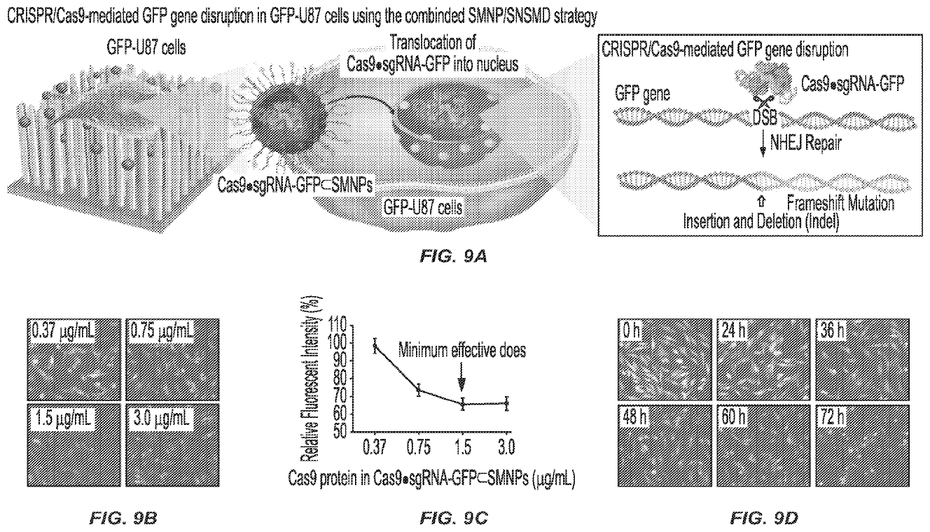

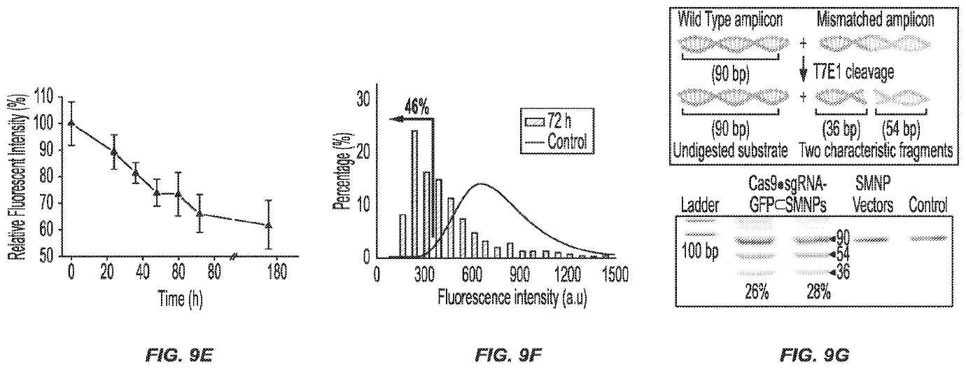

To explore the gene-editing utility of the combined SMNP/SNSMD strategy, its utility for CRISPR/Cas9-mediated GFP gene disruption in GFP-U87 cells was examined (FIG. 9A). A GFP-targeting sgRNA (sgRNA-GFP) was prepared according to literature.⁵⁹ Using the optimized formulation condition, the Cas9·sgRNA-GFP complex was encapsulated into a SMNP vector to give 8%-TAT-grafted Cas9·sgRNA-GFP⊂SMNPs (hydrodynamic sizes=120±30 nm). Successful introduction of Cas9·sgRNA-GFP into the GFP-U87 cells could disrupt GFP signals as a result of in frameshift mutation, which is induced by CRISPR/Cas9-mediated DNA double-strand breaks (DSB), followed by nucleotides deletion or insertion, and DNA repair via an endogenous non-homologous end joining (NHEJ) pathway.⁵⁹ First, it was examined how different doses of Cas9·sgRNA-GFP⊂SMNPs affected the GFP-disruption performance of the combined SMNP/SNSMD strategy. The EGFP-Cas9·sgRNA⊂SMNPs (containing 0.375, 0.75, 1.5, and 3.0 μg Cas9 protein) was first added to a culture well, in which an Ad-SiNWS (1×1 cm²) was immersed with 1.0 mL of DMEM medium. After settling growth-synchronized GFP-U87 cells onto Ad-SiNWS (loaded with Cas9·sgRNA-GFP⊂SMNPs) for 48 h, the cells were fixed for DAPI nuclear staining. The GFP-U87 cells on Ad-SiNWS were then subjected to microscopy imaging and image analysis to quantify residual EGFP signals. FIG. 9B compiles fluorescent micrographs of the GFP-U87 cells in the presence of different doses of Cas9·sgRNA-GFP⊂SMNPs. FIG. 9C summarizes the dose-dependent GFP disruption data, suggesting a minimum effective dose of Cas9 protein is 1.5 μg mL⁻¹. Using this minimum effective dose, we carried out time-dependent CRISPR/Cas9-mediated GFP disruption experiments to establish the correlation between GFP signal decay and treatment times. FIG. 9D compiles serial fluorescent micrographs of the GFP-U87 cells after settling onto Ad-SiNWS (loaded with Cas9·sgRNA-GFP⊂SMNPs) for 0, 24, 36, 48, 60, and 72 h. FIG. 9E shows the dose-dependent GFP disruption data, suggesting irreversible disruption of EGFP signals in the GFP-U87 cells from 0 to 72 h post cell settlement. Single-cell image analysis (FIG. 9F) unveiled that GFP disruption was successfully achieved in 46% of GFP-U87 cells. The GFP-U87 cells were cultured for another two weeks and the averaged GFP signals remains at a similar level. In an attempt to directly examine the CRISPR/Cas9-mediated gene disruption in GFP-U87 cells, the T7 endonuclease I (T7E1) assay was used to detect the insertion and deletion (indel) events associated with the Cas9 mediated gene editing at the genomic DNA level.⁶⁰ Genomic DNA was first extracted from treated GFP-U87 cells and then the sgRNA-targeted surrounding region was amplified via polymerase chain reaction (PCR). T7 endonuclease specifically recognizes and cleaves mismatched DNA amplicons associated with the Indel events (FIG. 9G). Along with the wild-type (WT) amplicon (90 bp), two characteristic fragments (330 bp and 244 bp) were detected and quantified by electrophoretogram (FIG. 9G). The intensity of DNA fragments was quantitated using ImageJ, revealing the indel efficiency based on the T7E1 assay was ˜27% in Cas9·sgRNA-GFP⊂SMNPs treated cells. The functional assay of GFP disruption revealed an approximately 46% CRISPR/Cas9-mediated gene editing efficiency whereas the T7E1 assay showed an approximately 27% efficiency at the genomic DNA level. The apparent discrepancy may be due to the T7E1 assay which has been shown to underestimate editing efficiency with a peak signal of 37% on a 50% mix of wildtype and mutant alleles.⁶¹

FIGS. 9A-9G are illustrations, fluorescent images, and data graphs showing CRISPR/Cas9-mediated GFP gene disruption in GFP-U87 cells using a combined SMNP/SNSMD strategy, according to an embodiment of the invention. Specifically, FIG. 9A is a schematic showing CRISPR/Cas9-mediated GFP gene disruption in GFP-U87 cells using the combined SMNP/SNSMD strategy. Cas9·sgRNA-GFP⊂SMNPs were prepared by co-encapsulating Cas9·sgRNA-GFP into a SMNP vector. FIG. 9B shows fluorescence micrographs of GFP-U87 cells collected at 48 h post GFP-U87 cell settlement. 4 Different Cas9 protein doses (0.375, 0.75, 1.5, and 3.0 μg/mL) in Cas9·sgRNA-GFP⊂SMNPs were studied. FIG. 9C is a graph showing quantitative analysis of the fluorescent micrographs in FIG. 9B showing dose-dependent disruption of the fluorescence signals in the GFP-U87 cells. A minimum effective dose is therefore determined as 1.5 μg of Cas9 protein per mL. FIG. 9D shows serial fluorescence micrographs of GFP-U87 cells collected at 0, 24, 36, 48, 60, 72, 170 h post GFP-U87 cell settlement. FIG. 9E shows a graph of quantitative analysis of the fluorescent micrographs in FIG. 9D, and shows time-dependent decay of GFP signals. FIG. 9F shows histograms of GFP singles in individual GFP-U87 cells treated by Cas9·sgRNA-GFP⊂SMNPs (20 μg/mL) after 72 h, suggesting successful disruption of GFP gene was achieved in 46% of GFP-U87 cells. FIG. 9G shows a T7E1 assay for the indel production efficiency of GFP gene in GFP-U87 cells treated by Cas9·sgRNA-GFP⊂SMNPs (1.5 μg/mL Cas9 protein) after 72 h.

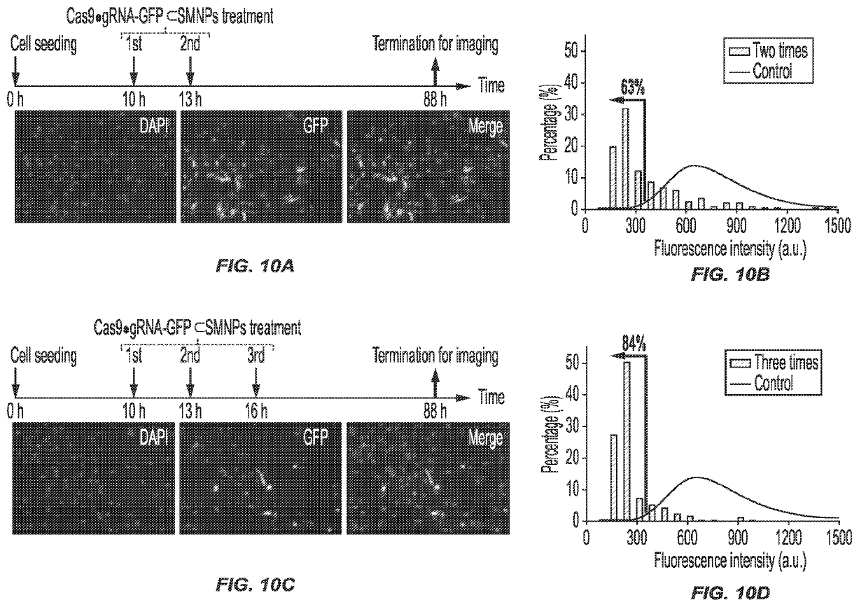

Introducing additional rounds of Cas9·sgRNA-GFP⊂SMNPs treatment after the initial GFP-U87 cell settlement could further increase the efficiency of CRISPR/Cas9-mediated GFP gene disruption (FIGS. 10A-10D). Based on the optimized condition, in which growth-synchronized GFP-U87 cells were settled onto Ad-SiNWS (loaded with 8% TAT-grafted Cas9·sgRNA-GFP⊂SMNPs, two or three rounds of 8% TAT-grafted Cas9·sgRNA-GFP⊂SMNPs (containing 1.5 μg Cas9 protein) were sequentially added to individual wells every 3 h to sustain a steady supply of Cas9·sgRNA-GFP complex. The treated cells were maintained in the same wells until termination at 88 h, and then observed via fluorescence microscopy. More GFP-U87 cells treated by Cas9·sgRNA-GFP⊂SMNPs for three times reduced their fluorescence signals (FIG. 10A), compared with the cells treated by one time (FIG. 9C) and two times (FIG. 10C). The fluorescence intensity distribution showed triple treatments of Cas9·sgRNA-GFP⊂SMNPs induce a higher gene disruption (84%), compared with one treatment (46%) and double treatment (63%) (FIGS. 10B and 10D), suggesting that the utility of the combined SMNP/SNSMD strategy for co-delivery of Cas9 protein and sgRNA via additional rounds of Cas9·sgRNA-GFP⊂SMNPs treatment, enables increase the efficiency of CRISPR/Cas9-mediated GFP gene disruption.

FIGS. 10A-10D are fluorescent images and data graphs showing results of two sequential treatments of Cas9·sgRNA-GFP⊂SMNPs to GFP-U87 cells via the combined SMNP/SNSMD strategy, according to an embodiment of the invention. Specifically, FIG. 10A shows the designated timelines summarize the two sequential treatments of Cas9·sgRNA-GFP⊂SMNPs to GFP-U87 cells via the combined SMNP/SNSMD strategy, sustaining a steady supply of Cas9·sgRNA-GFP. Fluorescence microscopy images of GFP-U87 cells double-treated by Cas9·sgRNA-GFP⊂SMNPs. Cells were visualized with DAPI staining and GFP expression. FIG. 10B shows histograms of GFP gene disruption performance in individual GFP-U87 cells double-treated by Cas9·sgRNA-GFP⊂SMNPs after 88 h. FIG. 10C shows the designated timelines and fluorescence microscopy images summarize the three sequential treatments of Cas9·sgRNA-GFP⊂SMNPs to GFP-U87 cells. FIG. 10D shows histograms of GFP gene disruption performance in individual GFP-U87 cells triple-treated by Cas9·sgRNA-GFP⊂SMNPs after 88 h.

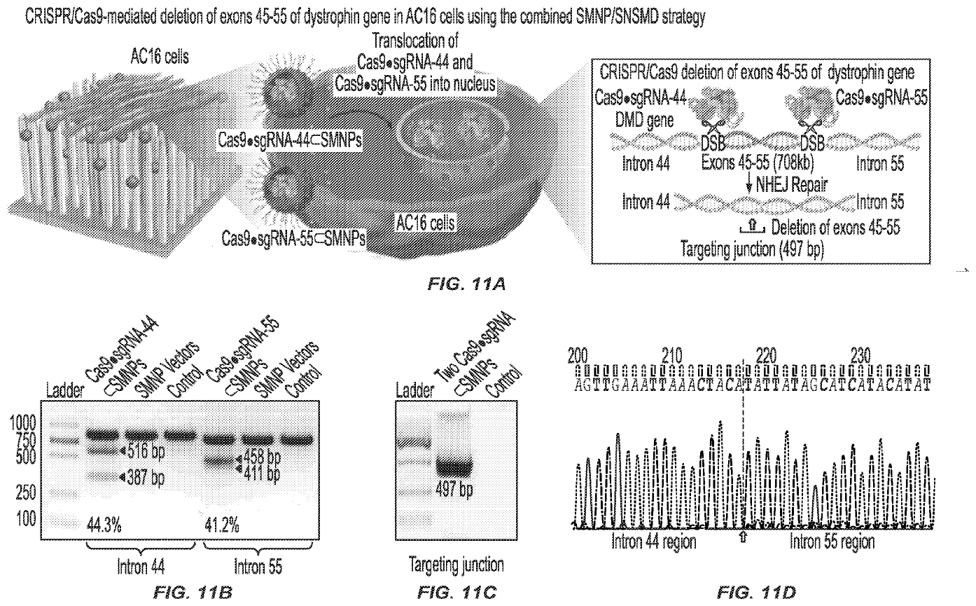

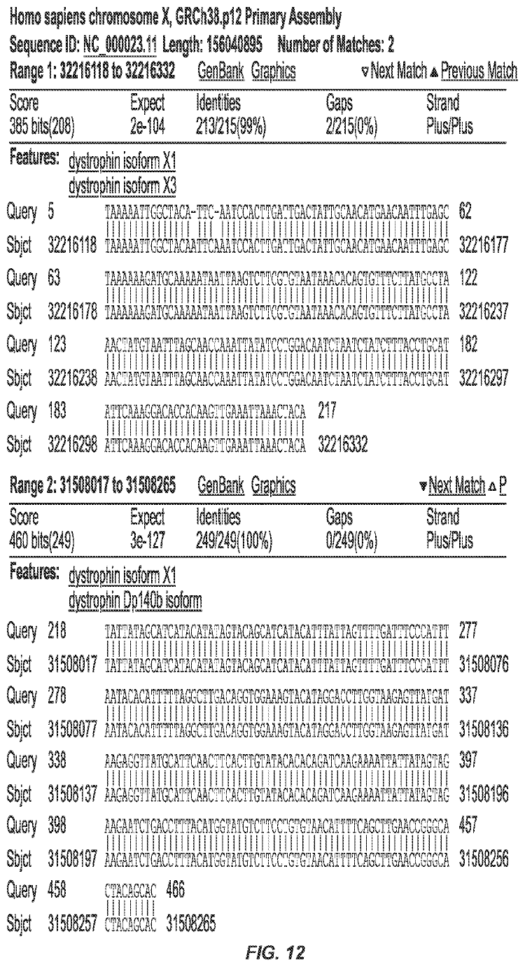

After successful demonstration of the combined SMNP/SNSMD strategy for i) co-delivery Cas9 and sgRNA and ii) GFP gene disruption, whether or not this approach could be employed for genomic editing of a genetic disease, e.g., DMD, was evaluated. DMD is a severe devastating muscle disease, affecting approximately 1 out of 5000 newborn males.⁵³ It is caused by mutations in the dystrophin gene, which could disrupt the open reading frame and thus ablate DMD protein translation, thereby causing a wide range of physical consequences. Although DMD is one of the most difficult genetic diseases to treat, and the dystrophin gene is the largest gene described in the human genome,⁶² about 60% of mutations causing DMD occur within exons 45-55 of dystrophin gene. Such mutations are often one or more exons deletions in the dystrophin gene, disrupting the reading frame of the gene and thus lead to a functional loss of dystrophin expression.⁶³ The potential for dystrophin gene modification using the CRISPR/Cas9 system has previously been reported in vitro and in vivo using viral vectors.⁶⁴⁻⁶⁷ For example, a pair of sgRNAs, i.e., sgRNA-44 and sgRNA-55, was designed and employed to achieve deletion of exons 45-55 (where 60% of DMD the mutations present). Through NHEJ pathway, the 3′ end of intron 44 and the 5′ end of intron 55 joined together, resulting in functional rescue of dystrophic phenotype.⁶⁶ However, this platform relied on viral vectors, which has limitations of packaging capacity and safety concerns of insertional mutagenesis. To address these safety concerns, the combined SMNP/SNSMD system for CRISPR/Cas9-mediated deletion of exons 45-55 of dystrophin gene in human cardiomyocytes AC16 cells (FIG. 11A) was used. Such a cell line is selected due to the fact about 90% DMD patients suffer from the cardiomyopathies, which are emerging as a main cause of morbidity and mortality.⁵⁴ First, two different SMNP vectors, i.e., 8% TAT-grafted Cas9·sgRNA-44⊂SMNPs (hydrodynamic sizes=110±20 nm) and 8% TAT-grafted Cas9·sgRNA-55⊂SMNPs (hydrodynamic sizes=120±25 nm) were prepared to encapsulate Cas9·sgRNA-44 (targeting introns 44) and Cas9·sgRNA-55 (targeting introns 55), respectively. Using the optimal protocol, the Cas9·sgRNA-44⊂SMNPs (containing 1.5 μg Cas9 protein) and Cas9·sgRNA-55⊂SMNPs (containing 1.5 μg Cas9 protein) were sequentially added to individual well every 3 h for three times. The SMNP vectors (no Cas9 protein) and control group were conducted in parallel. At 88 h post-treatment, the AC16 cells were harvested to analyze the gene indel production efficiency by T7E1 assay. As displayed in FIG. 11B, the indel production efficiency of Cas9·sgRNA-44⊂SMNPs and Cas9·sgRNA-55⊂SMNPs was 44.3% for intron 44 (two characteristic fragments are 516 and 387 bp) and 41.2% for intron 55 (two characteristic fragments are 458 and 411 bp), respectively. Furthermore, in order to confirm the deletion of exons 44-55, we used PCR method to detect the targeting DNA junction. First, the forward primer was designed at −231 bp for sgRNA-44 targeting sequence, i.e, intron 44, while the reverse primer was designed at +232 bp for sgRNA-55 targeting sequence, i.e, intron 55. Then by using PCR protocol, we successfully obtained a 497 bp targeting junction in the test group (Cas9·sgRNA-44⊂SMNPs and Cas9·sgRNA-55⊂SMNPs treatments), while no fragment was detected in control group (FIG. 11C). Sanger sequencing (FIG. 11D) was employed to analyze the DNA fragment at the targeting junction boundary (arrow in FIG. 11A and 11D), indicating precise deletion of exons 45-55 (708 kb) of dystrophin gene in AC16 cells using the combined SMNP/SNSMD strategy.

FIGS. 11A-11D are schematics and data graphs showing CRISPR/Cas9-mediated deletion of exons 45-55 of dystrophin gene in AC16 cells using a combined SMNP/SNSMD strategy, according to an embodiment of the invention. Specifically, FIG. 11A is a graphical summary for CRISPR/Cas9-mediated deletion of exons 45-55 of dystrophin gene in AC16 cells using the combined SMNP/SNSMD strategy. Two Cas9·sgRNA complexes (i.e., Cas9·sgRNA-44 targeting introns 44 and Cas9·sgRNA-55 targeting introns 55) were separately encapsulated in SMNP vectors to give Cas9·sgRNA-44⊂SMNPs and Cas9·sgRNA-55⊂SMNPs. FIG. 11B is a T7E1 assay for the indel production efficiency of DMD region deletion at introns 44 and 55 in AC16 cells triple-treated by Cas9·sgRNA-44⊂SMNPs and Cas9·sgRNA-55⊂SMNPs. FIG. 11C shows a PCR assay and FIG. 11D shows results of Sanger sequencing for confirming the deletion of exons 44-55 of dystrophin gene.

### CONCLUSIONS

The feasibility of applying the combined SMNP/SNSMD strategy to co-deliver CRISPR/Cas9 protein and sgRNA into target cells in a highly efficient manner has been demonstrated. The unique self-assembly synthetic strategy for the preparation of Cas9·sgRNA⊂SMNPs has to do with the combined use of multiple molecular recognition mechanisms. This SMNP/SNSMD strategy allows for dynamic assembly and local enrichment of Cas9·sgRNA⊂SMNPs from the surrounding solution/medium onto Ad-SiNWS. As cells settle onto the substrates, the juxtaposition between the cell membrane and the nanowires on Ad-SiNWS facilitates the uptake of Cas9·sgRNA⊂SMNPs into the cells. The Cas9 protein and sgRNA-GFP were successfully co-delivered into GFP-U87 cells leading to highly efficient disruption of GFP expression. Utilizing an optimized formulation, efficient deletion of exons 45-55 (up to 708 kb) in the dystrophin gene in AC16 cells by co-delivering a pair of Cas9·sgRNA complexes was achieved. This approach could be adopted in the study of the mechanism and therapeutic strategy of a wide spectrum of diseases. Future studies will focus on validating the feasibility of this system to co-deliver Cas9·sgRNA and DNA plasmid to correct mutated genes via a knock-in mode (as opposed to partial gene removal via a knock-out mode presented here), thus broadening the type of diseases that could be targeted by the approach.

Methods Section

Materials. Chemical reagents and solvents were purchased from Sigma and were used as received without further purification unless otherwise noted. Cas9 Nuclease: NLS-EGFP-Cas9 nuclease were purchased from Genscript company, while Cas9 protein was purchased from Takara company. For cell culture: the 24-well plates were purchased from Thermofisher. U87 (human brain glioblastoma cell line), and AC16 (human cardiac cell lines) were both purchased from American Type Culture Collection (ATCC). Fetal bovine serum (FBS), Dulbeco's modified Eagle's medium (DMEM) and OPTI-MEM were obtained from Gibco (Life technology). For Indel analysis: DNA extraction kit was purchased from Qiagen (QIAamp® DNA Mini Kit). EnGen® Mutation Detection kit was purchased from NEB.

Measurement. Dynamic light scattering of Cas9·sgRNA⊂SMNPs was measured on Zetasizer Nano instrument (Malvern Instruments Ltd., United Kingdom). Cell imaging studies were performed on a Nikon TE2000S inverted fluorescent microscope with a cooled charge-coupled device (CCD) camera (QImaging, Retiga 4000R), X-Cite 120 Mercury lamp, automatic stage, and filters for five fluorescent channels (W1: 325-375 nm, W2: 465-495 nm, W3: 570-590 nm, W4: 590-650 nm, and W5: 650-900 nm). Fluorescence intensities were measured by a Fujifilm BAS-5000 microplate reader. SEM images were performed on a TS-5136MM (TESCAN, Czech) scanning electron microscope at an accelerating voltage of 20 kV. Samples dispersed at an appropriate concentration were cast onto a glass sheet at room temperature and sputter-coated with gold.

Preparation of Adamantane-Grafted Silicon Nanowire Substrates (Ad-SiNWS). SiNWS were fabricated via a wet chemical etching process. First, the surface of the silicon substrate was made hydrophilic according to the following procedure: the silicon wafer was ultrasonicated in acetone and ethanol at room temperature for 10 and 5 min, respectively, to remove contamination from organic grease. Then, the degreased silicon substrate was heated in boiling piranha solution (4:1 (v/v) H₂SO₄/H₂O₂) and RCA solution (1:1:5 (v/v/v) NH₃/H₂O₂/H₂O) each for 1 h. Subsequently, the silicon substrate was rinsed several times with deionized water. Then, the clean silicon substrate was used in a wet chemical etching process. An etching mixture consisting of deionized water, 4.6 M HF, and 0.2 M silver nitrate was used at room temperature. The etching duration was dependent upon the required length of the nanowires. After etching, the substrate was immersed in boiling aqua regia (3:1 (v/v) HCl/HNO₃) for 15 min to remove the silver film. Finally, the substrate was rinsed with DI water and dried under nitrogen and was then ready for surface modification. The surface modification of the SiNWS was processed with 4% (v/v) 3-aminopropyl trimethoxysilane in ethanol at room temperature for 45 min. Then, the SiNWS were treated with the 1-adamantane isocyanate (1.0 mM) in DMSO for 30 min. The modified Ad-SiNWS were then washed with DMSO twice to remove excess 1-adamantane isocyanate. The substrates were rinsed with DI water three times and stored at 4° C. before cell seeding. The diameters and lengths of Ad-SiNWS were 100-150 nanometers and 5-10 micrometers, respectively.

SgRNA Synthesis. Briefly, the targeting sequence of sgRNA-GFP is GCCGTCCAGCTCGACCAGGA (SEQ ID NO:1). GFP sgRNAs were synthesized by Biosynthesis company. The sgRNA sequences of DMD are: sgRNA-44: GTTGAAATTAAACTACACACTGG (SEQ ID NO:2); sgRNA-55: TGTATGATGCTATAATACCAAGG (SEQ ID NO:3). The sequence of sgRNA-44 and sgRNA-55 was designed according to Young et al.⁶⁷ and synthesized by Synbio Technologies company (Suzhou, China).

Synthesis of EGFP-Cas9·sgRNA⊂SMNPs. A self-assembly procedure was applied to prepare the EGFP-Cas9 protein and sgRNA encapsulated supramolecular nanoparticles (EGFP-Cas9·sgRNA⊂SMNPs). Three batches of EGFP-Cas9·sgRNA⊂SMNPs (FIG. 7B) were formulated via systemically modulating i) the weight ratios (wt %) between SMNP vector and EGFP-Cas9·sgRNA payload, ii) sizes of Cas9·sgRNA⊂SMNPs, and iii) the percentages of TAT ligand on SMNP surfaces. The optimal synthesis formulation is below: A total of 2.0 μL DMSO solution containing Ad-PAMAM (0.42 μg) was added into a 50 μL PBS mixture with EGFP-Cas9 protein (6.0 μg), sgRNA (1.2 μg, Mol ratio≈1:1), Ad-PEG (55 μg), CD-PEI (21 μg), and Ad-PEG-TAT (6.0 μg). The above resulting mixture was then stirred vigorously to achieve optimal Cas9·sgRNA⊂SMNPs. The mixture was stored at 4° C. for 30 min, after that, dynamic light scattering (DLS) and scaning electron microscope (SEM) were used to character the sizes of EGFP-Cas9·sgRNA⊂SMNPs.

Delivery EGFP-Cas9·sgRNA⊂SMNPs to U87 cells via the combined SMNP/SNSMD strategy. Prior to settling the cells onto Ad-SiMWS, U87 cells were starved in serum-free DMEM overnight (10 h) to synchronize cells to G0/G1 phases of cell cycle.⁵⁸ A 1×10⁵ mount of U87 were introduced into each well of a 24-well plate, in which a 1×1 cm² Ad-SiNWS loading with different concentrations of EGFP-Cas9·sgRNA⊂SMNPs was placed in. The cells were co-incubated with for the designated time point in the test group. The PBS was added in the control groups. After washing with PBS, the cells in chamber were immediately fixed with 2% PFA, and then stained with DAPI. Microscopy-based image cytometry was used to detect the deliver performances of different conditions. After different treatments, the cells were harvested and the GFP signal was quantified with fluorescent microscope with a CCD camera (Nilon H550, Japan).

Synthesis of Cas9·sgRNA-GFP⊂SMNPs. A total of 2.0 μL DMSO solution containing Ad-PAMAM (0.42 μg) was added into a 50 μL PBS mixture with Cas9 protein (6.0 μg), sgRNA-GFP (1.2 μg, Mol ratio≈1:1), Ad-PEG (55 μg), CD-PEI (21 μg), and Ad-PEG-TAT (6.0 μg). The above resulting mixture was then stirred vigorously to achieve optimal Cas9·sgRNA⊂SMNPs. The mixture was stored at 4° C. for 30 min, after that, DLS was used to character the sizes of Cas9·sgRNA-GFP⊂SMNPs.

GFP gene disruption assay on GFP-expressing U87 cells (GFP-U87). A 1×10⁵mount of GFP-U87 (starved in serum-free DMEM for 10 h) were introduced into each well of a 24-well plate, in which a 1×1 cm² Ad-SiNWS loading with different concentrations of Cas9·sgRNA-GFP⊂SMNPs was placed in. The cells were co-incubated with for the designated time point in the test group. The PBS was added in the control groups. After washing with PBS, the cells in chamber were immediately fixed with 2% PFA, and then stained with DAPI. Microscopy-based image cytometry was used to detect the disruption performances of different conditions. After different treatments, the cells were harvested and the GFP signal was quantified with fluorescent microscope with a CCD camera (Nilon H550, Japan).

Synthesis of Cas9·sgRNA-44⊂SMNPs and Cas9·sgRNA-55⊂SMNPs. A total of 2.0 μL DMSO solution containing Ad-PAMAM (0.42 μg) was added into a 50 μL PBS mixture with Cas9 protein (6.0 μg), sgRNA-44 or sgRNA-55 (1.2 μg), Ad-PEG (55 μg), CD-PEI (21 μg), and Ad-PEG-TAT (6.0 μg). The above resulting mixture was then stirred vigorously to achieve Cas9·sgRNA-44⊂SMNPs and Cas9·sgRNA-55⊂SMNPs. The mixture was stored at 4° C. for 30 min, after that, DLS was used to character the sizes of Cas9·sgRNA-44⊂SMNPs and Cas9·sgRNA-55⊂SMNPs.

DMD gene deletion assay in AC16 human cardiomyocytes. A 1×10⁵ mount of AC16 cells were introduced into each well of a 24-well plate, in which a 1×1 cm² Ad-SiNWS was placed in. The Cas9·sgRNA-44⊂SMNPs (20 μg mL⁻¹ in 1 mL of DMEM medium) and Cas9·sgRNA-55⊂SMNPs (20 μg mL⁻¹ in 1 mL of DMEM medium) were sequentially added to individual well every 3 h for three times. Two control studies were conducted using SMNP vectors and PBS solution. After 48 h post-treatment, the cells were harvested to analyze the gene indel production efficiency by T7E1 assay and PCR assay.

DNA extraction and polymerase chain reaction (PCR). After Cas9·sgRNA⊂SMNPs delivery for different times, cells were washed with PBS and replaced with DMEM, and then allowed to grow for 48 h. At this time point, cells were harvested, and genomic DNA was extracted with a commercial QIAamp® DNA Mini Kit (Qiagen, Germany), following manufacturer's instructions. Then, PCR was conducted to amplify GFP and DMD. The primer sequences were listed as follow: GFP: forward: 5′-GAGCAAGGGCGAGGAGC-3′ (SEQ ID NO:4), reverse: 5′-CCGGACACGCTGAACTTGTG-3′ (SEQ ID NO:5). DMD introns-44: forward: 5′-GAGAGTTTGCCTGGACGGA-3′ (SEQ ID NO:6), reverse: 5′-CCTCTCTATACAAATGCCAACGC-3′ (SEQ ID NO:7). DMD introns-55: forward: 5′-TCCAGGCCTCCTCTCTTTGA-3′ (SEQ ID NO:8), reverse: 5′-CCCTTTTCTTGGCGTATTGCC-3′ (SEQ ID NO:9).

GFP and DMD were amplified with a S1000™ Thermal Cycler (Bio-Rad) under the following PCR conditions: 95° C. for 3 minutes followed by 35 cycles (95° C. for 15″, 58° C. for 15″ and 72° C. for 20″) and 72° C. for 3 minutes. The PCR products were checked on a 1.5% electrophoresis gel.

T7 endonuclease assay. After amplification, the PCR products were hybridized and digested with a T7 endonuclease 1 mutation detection kit (New England Biolabs, NEB #E3321)assays kit. After incubation at 37° C. for 30 minutes, The product from T7E1 assay was run on a 15% acrylamide gel stained with ethidium bromide, and the bands was analyzed by a gel imaging instrument.

PCR for exons 44-55 deletion in the DMD gene. Primer introns-44 forward: 5′-TGCCCTCATCTGTCTTAATCAGTA-3′ (SEQ ID NO:10). Primer introns-55 reverse: 5′-GTGCTGTAGTGCCCGGTT-3′ (SEQ ID NO:11), Primers were synthesized by Sangon biotech company(Shanghai, China). The target sequence for deletion was amplified with a S1000™ Thermal Cycler (Bio-Rad) under the following PCR conditions: 95° C. for 5 minutes followed by 35 cycles (95° C. for 15″, 58° C. for 15″ and 72° C. for 30″) and 72° C. for 3 minutes. The PCR products were checked on a 1.5% electrophoresis gel. When electrophoresis was completed, a longitudinal slice of the gel was cut, fixed, and purified to extract DNA using a FastPure Gel DNA Extraction Mini Kit (Vazyme, China). The DNA was sent to Sangon biotech company (Shanghai, China) for sequencing.

DNA Sequence for exons 44-55 deletion in the DMD gene:

Sanger sequencing was employed to analyze the DNA fragment (466 bp) containing the repaired junction boundary, and then compared the sequence using BLAST (basic local alignment search tool) program. The results displayed the sequence matched to two ranges in dystrophin gene, i.e., range1: 32216118 to 32216322 and range 2: 31508017 to 31508265, indicating precise deletion of exons 45-55 (708 kb) of dystrophin gene in AC16 cells.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

### REFERENCES FROM EXAMPLE 2

1. Ran, F. A.; Hsu, P. D.; Wright, J.; Agarwala, V.; Scott, D. A.; Zhang, F. Genome Engineering Using the Crispr-Cas9 System. Nat. Protoc. 2013, 8, 2281.
2. Doudna, J. A.; Charpentier, E. The New Frontier of Genome Engineering with Crispr-Cas9. Science 2014, 346, 1258096.
3. Jiang, F.; Doudna, J. A. Crispr-Cas9 Structures and Mechanisms. Annual review of biophysics 2017, 46, 505-529.
4. Nishimasu, H.; Ran, F. A.; Hsu, P. D.; Konermann, S.; Shehata, S. I.; Dohmae, N.; Ishitani, R.; Zhang, F.; Nureki, O. Crystal Structure of Cas9 in Complex with Guide Rna and Target DNA. Cell 2014, 156, 935-949.
5. Sternberg, S. H.; Redding, S.; Jinek, M.; Greene, E. C.; Doudna, J. A. DNA Interrogation by the Crispr Rna-Guided Endonuclease Cas9. Nature 2014, 507, 62-67.
6. Sternberg, S. H.; LaFrance, B.; Kaplan, M.; Doudna, J. A. Conformational Control of DNA Target Cleavage by Crispr-Cas9. Nature 2015, 527, 110-113.
7. Hsu, P. D.; Lander, E. S.; Zhang, F. Development and Applications of Crispr-Cas9 for Genome Engineering. Cell 2014, 157, 1262-1278.
8. Wang, H.; Yang, H.; Shivalila, C. S.; Dawlaty, M. M.; Cheng, A. W.; Zhang, F.; Jaenisch, R. One-Step Generation of Mice Carrying Mutations in Multiple Genes by Crispr/Cas-Mediated Genome Engineering. Cell 2013, 153, 910-918.
9. Santiago-Fernandez, O.; Osorio, F. G.; Quesada, V.; Rodriguez, F.; Basso, S.; Maeso, D.; Rolas, L.; Barkaway, A.; Nourshargh, S.; Folgueras, A. R. Development of a Crispr/Cas9-Based Therapy for Hutchinson-Gilford Progeria Syndrome. Nat. Med. 2019, 25, 423.
10. Niu, Y.; Shen, B.; Cui, Y.; Chen, Y.; Wang, J.; Wang, L.; Kang, Y.; Zhao, X.; Si, W.; Li, W. Generation of Gene-Modified Cynomolgus Monkey Via Cas9/Rna-Mediated Gene Targeting in One-Cell Embryos. Cell 2014, 156, 836-843.
11. Long, C.; Amoasii, L.; Mireault, A. A.; McAnally, J. R.; Li, H.; Sanchez-Ortiz, E.; Bhattacharyya, S.; Shelton, J. M.; Bassel-Duby, R.; Olson, E. N. Postnatal Genome Editing Partially Restores Dystrophin Expression in a Mouse Model of Muscular Dystrophy. Science 2016, 351, 400-403.
12. Tabebordbar, M.; Zhu, K.; Cheng, J. K.; Chew, W. L.; Widrick, J. J.; Yan, W. X.; Maesner, C.; Wu, E. Y.; Xiao, R.; Ran, F. A. In Vivo Gene Editing in Dystrophic Mouse Muscle and Muscle Stem Cells. Science 2016, 351, 407-411.
13. Nelson, C. E.; Hakim, C. H.; Ousterout, D. G.; Thakore, P. I.; Moreb, E. A.; Rivera, R. M. C.; Madhavan, S.; Pan, X.; Ran, F. A.; Yan, W. X. In Vivo Genome Editing Improves Muscle Function in a Mouse Model of Duchenne Muscular Dystrophy. Science 2016, 351, 403-407.
14. Nelson, C. E.; Wu, Y.; Gemberling, M. P.; Oliver, M. L.; Waller, M. A.; Bohning, J. D.; Robinson-Hamm, J. N.; Bulaklak, K.; Rivera, R. M. C.; Collier, J. H. Long-Term Evaluation of Aav-Crispr Genome Editing for Duchenne Muscular Dystrophy. Nat. Med. 2019, 25, 427.
15. Maeder, M. L.; Stefanidakis, M.; Wilson, C. J.; Baral, R.; Barrera, L. A.; Bounoutas, G. S.; Bumcrot, D.; Chao, H.; Ciulla, D. M.; DaSilva, J. A. Development of a Gene-Editing Approach to Restore Vision Loss in Leber Congenital Amaurosis Type 10. Nat. Med. 2019, 25, 229.
16. Lucky, S. S.; Soo, K. C.; Zhang, Y. Nanoparticles in Photodynamic Therapy. Chem. Rev. 2015, 115, 1990-2042.
17. Gori, J. L.; Hsu, P. D.; Maeder, M. L.; Shen, S.; Welstead, G. G.; Bumcrot, D. Delivery and Specificity of Crispr/Cas9 Genome Editing Technologies for Human Gene Therapy. Human gene therapy 2015, 26, 443-451.
18. Wang, H.-X.; Li, M.; Lee, C. M.; Chakraborty, S.; Kim, H.-W.; Bao, G.; Leong, K. W. Crispr/Cas9-Based Genome Editing for Disease Modeling and Therapy: Challenges and Opportunities for Nonviral Delivery. Chem. Rev. 2017, 117, 9874-9906.
19. Sanjana, N. E.; Shalem, O.; Zhang, F. Improved Vectors and Genome-Wide Libraries for Crispr Screening. Nature methods 2014, 11, 783-784.
20. Maggio, I.; Holkers, M.; Liu, J.; Janssen, J. M.; Chen, X.; Goncalves, M. A. Adenoviral Vector Delivery of Rna-Guided Crispr/Cas9 Nuclease Complexes Induces Targeted Mutagenesis in a Diverse Array of Human Cells. Sci Rep 2014, 4, 5105.
21. Senis, E.; Fatouros, C.; GroBe, S.; Wiedtke, E.; Niopek, D.; Mueller, A. K.; Borner, K.; Grimm, D. Crispr/Cas9-Mediated Genome Engineering: An Adeno-Associated Viral (Aav) Vector Toolbox. Biotechnology journal 2014, 9, 1402-1412.
22. Swiech, L.; Heidenreich, M.; Banerjee, A.; Habib, N.; Li, Y.; Trombetta, J.; Sur, M.; Zhang, F. In Vivo Interrogation of Gene Function in the Mammalian Brain Using Crispr-Cas9. Nature biotechnology 2015, 33, 102-106.
23. Wu, Z.; Yang, H.; Colosi, P. Effect of Genome Size on Aav Vector Packaging. Mol. Ther. 2010, 18, 80-86.
24. Li, L.; Hu, S.; Chen, X. Non-Viral Delivery Systems for Crispr/Cas9-Based Genome Editing: Challenges and Opportunities. Biomaterials 2018, 171, 207-218.
25. Zuris, J. A.; Thompson, D. B.; Shu, Y.; Guilinger, J. P.; Bessen, J. L.; Hu, J. H.; Maeder, M. L.; Joung, J. K.; Chen, Z.-Y.; Liu, D. R. Cationic Lipid-Mediated Delivery of Proteins Enables Efficient Protein-Based Genome Editing in Vitro and in Vivo. Nat. Biotechnol. 2015, 33, 73.
26. Wang, M.; Zuris, J. A.; Meng, F.; Rees, H.; Sun, S.; Deng, P.; Han, Y.; Gao, X.; Pouli, D.; Wu, Q. Efficient Delivery of Genome-Editing Proteins Using Bioreducible Lipid Nanoparticles. Proceedings of the National Academy of Sciences 2016, 113, 2868-2873.
27. Li, L.; Song, L.; Liu, X.; Yang, X.; Li, X.; He, T.; Wang, N.; Yang, S.; Yu, C.; Yin, T. Artificial Virus Delivers Crispr-Cas9 System for Genome Editing of Cells in Mice. ACS Nano 2016, 11, 95-111.
28. Sun, W.; Ji, W.; Hall, J. M.; Hu, Q.; Wang, C.; Beisel, C. L.; Gu, Z. Self-Assembled DNA Nanoclews for the Efficient Delivery of Crispr-Cas9 for Genome Editing. Angew. Chem. Int. Ed. 2015, 54, 12029-12033.
29. Liu, Q.; Zhao, K.; Wang, C.; Zhang, Z.; Zheng, C.; Zhao, Y.; Zheng, Y.; Liu, C.; An, Y.; Shi, L. Multistage Delivery Nanoparticle Facilitates Efficient Crispr/Dcas9 Activation and Tumor Growth Suppression in Vivo. Advanced Science 2019, 6.
30. Zhou, W.; Cui, H.; Ying, L.; Yu, X. F. Enhanced Cytosolic Delivery and Release of Crispr/Cas9 by Black Phosphorus Nanosheets for Genome Editing. Angew. Chem. 2018, 130, 10425-10429.
31. Wang, P.; Zhang, L.; Zheng, W.; Cong, L.; Guo, Z.; Xie, Y.; Wang, L.; Tang, R.; Feng, Q.; Hamada, Y. Thermo-Triggered Release of Crispr-Cas9 System by Lipid-Encapsulated Gold Nanoparticles for Tumor Therapy. Angew. Chem. Int. Ed. 2018, 57, 1491-1496.
32. Lee, B.; Lee, K.; Panda, S.; Gonzales-Rojas, R.; Chong, A.; Bugay, V.; Park, H. M.; Brenner, R.; Murthy, N.; Lee, H. Y. Nanoparticle Delivery of Crispr into the Brain Rescues a Mouse Model of Fragile X Syndrome from Exaggerated Repetitive Behaviours. Nature biomedical engineering 2018, 2, 497.
33. Lee, K.; Conboy, M.; Park, H. M.; Jiang, F.; Kim, H. J.; Dewitt, M. A.; Mackley, V. A.; Chang, K.; Rao, A.; Skinner, C. Nanoparticle Delivery of Cas9 Ribonucleoprotein and Donor DNA in Vivo Induces Homology-Directed DNA Repair. Nature biomedical engineering 2017, 1, 889.
34. Li, L.; Yang, Z.; Zhu, S.; He, L.; Fan, W.; Tang, W.; Zou, J.; Shen, Z.; Zhang, M.; Tang, L. A Rationally Designed Semiconducting Polymer Brush for Nir-li Imaging-Guided Light-Triggered Remote Control of Crispr/Cas9 Genome Editing. Adv. Mater. 2019, 31, 1901187.
35. Miller, J. B.; Zhang, S.; Kos, P.; Xiong, H.; Zhou, K.; Perelman, S. S.; Zhu, H.; Siegwart, D. J. Non-Viral Crispr/Cas Gene Editing in Vitro and in Vivo Enabled by Synthetic Nanoparticle Co-Delivery of Cas9 Mrna and Sgrna. Angew. Chem. Int. Ed. 2017, 56, 1059-1063.
36. Jiang, C.; Mei, M.; Li, B.; Zhu, X.; Zu, W.; Tian, Y.; Wang, Q.; Guo, Y.; Dong, Y.; Tan, X. A Non-Viral Crispr/Cas9 Delivery System for Therapeutically Targeting Hbv DNA and Pcsk9 in Vivo. Cell Res. 2017, 27, 440.
37. Finn, J. D.; Smith, A. R.; Patel, M. C.; Shaw, L.; Youniss, M. R.; van Heteren, J.; Dirstine, T.; Ciullo, C.; Lescarbeau, R.; Seitzer, J. A Single Administration of Crispr/Cas9 Lipid Nanoparticles Achieves Robust and Persistent in Vivo Genome Editing. Cell reports 2018, 22, 2227-2235.
38. Liu, J.; Chang, J.; Jiang, Y.; Meng, X.; Sun, T.; Mao, L.; Xu, Q.; Wang, M. Fast and Efficient Crispr/Cas9 Genome Editing in Vivo Enabled by Bioreducible Lipid and Messenger Rna Nanoparticles. Adv. Mater. 2019, 1902575.
39. Gao, X.; Tao, Y.; Lamas, V.; Huang, M.; Yeh, W.-H.; Pan, B.; Hu, Y.-J.; Hu, J. H.; Thompson, D. B.; Shu, Y. Treatment of Autosomal Dominant Hearing Loss by in Vivo Delivery of Genome Editing Agents. Nature 2018, 553, 217.
40. Hansen-Bruhn, M.; de Avila, B. E. F.; Beltrán-Gastélum, M.; Zhao, J.; Ramirez-Herrera, D. E.; Angsantikul, P.; Vesterager Gothelf, K.; Zhang, L.; Wang, J. Active Intracellular Delivery of a Cas9/Sgma Complex Using Ultrasound-Propelled Nanomotors. Angew. Chem. Int. Ed. 2018, 57, 2657-2661.
41. Alsaiari, S. K.; Patil, S.; Alyami, M.; Alamoudi, K. O.; Aleisa, F. A.; Merzaban, J. S.; Li, M.; Khashab, N. M. Endosomal Escape and Delivery of Crispr/Cas9 Genome Editing Machinery Enabled by Nanoscale Zeolitic Imidazolate Framework. J. Am. Chem. Soc. 2017, 140, 143-146.
42. Pan, Y.; Yang, J.; Luan, X.; Liu, X.; Li, X.; Yang, J.; Huang, T.; Sun, L.; Wang, Y.; Lin, Y. Near-Infrared Upconversion-Activated Crispr-Cas9 System: A Remote-Controlled Gene Editing Platform. Science Advances 2019, 5, eaav7199.
43. Shahbazi, R.; Sghia-Hughes, G.; Reid, J. L.; Kubek, S.; Haworth, K. G.; Humbert, O.; Kiem, H.-P.; Adair, J. E. Targeted Homology-Directed Repair in Blood Stem and Progenitor Cells with Crispr Nanoformulations. Nature materials 2019, 1.
44. Wang, H.; Wang, S.; Su, H.; Chen, K. J.; Armijo, A. L.; Lin, W. Y.; Wang, Y.; Sun, J.; Kamei, K. i.; Czernin, J. A Supramolecular Approach for Preparation of Size-Controlled Nanoparticles. Angewandte Chemie International Edition 2009, 48, 4344-4348.
45. Chen, K.-J.; Wolahan, S. M.; Wang, H.; Hsu, C.-H.; Chang, H.-W.; Durazo, A.; Hwang, L.-P.; Garcia, M. A.; Jiang, Z. K.; Wu, L. A Small Mn Contrast Agent Library of Gadolinium (Iii)-Encapsulated Supramolecular Nanoparticles for Improved Relaxivity and Sensitivity. Biomaterials 2011, 32, 2160-2165.
46. Choi, J.-s.; Zhu, Y.; Li, H.; Peyda, P.; Nguyen, T. T.; Shen, M. Y.; Yang, Y. M.; Zhu, J.; Liu, M.; Lee, M. M. Cross-Linked Fluorescent Supramolecular Nanoparticles as Finite Tattoo Pigments with Controllable Intradermal Retention Times. ACS Nano 2016, 11, 153-162.
47. Lee, J. H.; Chen, K. J.; Noh, S. H.; Garcia, M. A.; Wang, H.; Lin, W. Y.; Jeong, H.; Kong, B. J.; Stout, D. B.; Cheon, J. On-Demand Drug Release System for in Vivo Cancer Treatment through Self-Assembled Magnetic Nanoparticles. Angew. Chem. Int. Ed. 2013, 52, 4384-4388.
48. Wang, S.; Chen, K. J.; Wu, T. H.; Wang, H.; Lin, W. Y.; Ohashi, M.; Chiou, P. Y.; Tseng, H. R. Photothermal Effects of Supramolecularly Assembled Gold Nanoparticles for the Targeted Treatment of Cancer Cells. Angew. Chem. Int. Ed. 2010, 49, 3777-3781.
49. Peng, J.; Garcia, M. A.; Choi, J.-s.; Zhao, L.; Chen, K.-J.; Bernstein, J. R.; Peyda, P.; Hsiao, Y.-S.; Liu, K. W.; Lin, W.-Y. Molecular Recognition Enables Nanosubstrate-Mediated Delivery of Gene-Encapsulated Nanoparticles with High Efficiency. ACS nano 2014, 8, 4621-4629.
50. Hou, S.; Choi, J. s.; Chen, K. J.; Zhang, Y.; Peng, J.; Garcia, M. A.; Yu, J. H.; Thakore-Shah, K.; Ro, T.; Chen, J. F. Supramolecular Nanosubstrate-Mediated Delivery for Reprogramming and Transdifferentiation of Mammalian Cells. small 2015, 11, 2499-2504.
51. Liu, Y.; Du, J.; Choi, J. s.; Chen, K. J.; Hou, S.; Yan, M.; Lin, W. Y.; Chen, K. S.; Ro, T.; Lipshutz, G. S. A High-Throughput Platform for Formulating and Screening Multifunctional Nanoparticles Capable of Simultaneous Delivery of Genes and Transcription Factors. Angew. Chem. Int. Ed. 2016, 55, 169-173.
52. Liu, Y.; Wang, H.; Kamei, K. i.; Yan, M.; Chen, K. J.; Yuan, Q.; Shi, L.; Lu, Y.; Tseng, H. R. Delivery of Intact Transcription Factor by Using Self-Assembled Supramolecular Nanoparticles. Angewandte Chemie 2011, 123, 3114-3118.
53. Yiu, E. M.; Kornberg, A. J. Duchenne Muscular Dystrophy. J. Paediatr. Child Health 2015, 51, 759-764.
54. Spurney, C. F. Cardiomyopathy of Duchenne Muscular Dystrophy: Current Understanding and Future Directions. Muscle Nerve 2011, 44, 8-19.
55. Fayssoil, A.; Nardi, O.; Orlikowski, D.; Annane, D. Cardiomyopathy in Duchenne Muscular Dystrophy: Pathogenesis and Therapeutics. Heart Fail. Rev. 2010, 15, 103.
56. Béroud, C.; Tuffery-Giraud, S.; Matsuo, M.; Hamroun, D.; Humbertclaude, V.; Monnier, N.; Moizard, M. P.; Voelckel, M. A.; Calemard, L. M.; Boisseau, P. Multiexon Skipping Leading to an Artificial Dmd Protein Lacking Amino Acids from Exons 45 through 55 Could Rescue up to 63% of Patients with Duchenne Muscular Dystrophy. Human mutation 2007, 28, 196-202.
57. Peng, K. Q.; Yan, Y. J.; Gao, S. P.; Zhu, J. Synthesis of Large-Area Silicon Nanowire Arrays Via Self-Assembling Nanoelectrochemistry. Adv. Mater. 2002, 14, 1164-1167.
58. Golding, S. E.; Rosenberg, E.; Khalil, A.; McEwen, A.; Holmes, M.; Neill, S.; Povirk, L. F.; Valerie, K. Double Strand Break Repair by Homologous Recombination Is Regulated by Cell Cycle-Independent Signaling Via Atm in Human Glioma Cells. J. Biol. Chem. 2004, 279, 15402-15410.
59. Wang, M.; Zuris, J. A.; Meng, F.; Rees, H.; Sun, S.; Deng, P.; Han, Y.; Gao, X.; Pouli, D.; Wu, Q.; Georgakoudi, I.; Liu, D. R.; Xu, Q. Efficient Delivery of Genome-Editing Proteins Using Bioreducible Lipid Nanoparticles. Proc. Natl. Acad. Sci. U.S.A. 2016, 113, 2868-2873.
60. Schumann, K.; Lin, S.; Boyer, E.; Simeonov, D. R.; Subramaniam, M.; Gate, R. E.; Haliburton, G. E.; Chun, J. Y.; Bluestone, J. A.; Doudna, J. A. Generation of Knock-in Primary Human T Cells Using Cas9 Ribonucleoproteins. Proc. Natl. Acad. Sci. U.S.A. 2015, 112, 10437-10442.
61. Vouillot, L.; Thélie, A.; Pollet, N. Comparison of T7e1 and Surveyor Mismatch Cleavage Assays to Detect Mutations Triggered by Engineered Nucleases. G3: Genes, Genomes, Genetics 2015, g3. 114.015834.
62. Tennyson, C. N.; Klamut, H. J.; Worton, R. G. The Human Dystrophin Gene Requires 16 Hours to Be Transcribed and Is Cotranscriptionally Spliced. Nat. Genet. 1995, 9, 184.
63. Bushby, K.; Finkel, R.; Birnkrant, D. J.; Case, L. E.; Clemens, P. R.; Cripe, L.; Kaul, A.; Kinnett, K.; McDonald, C.; Pandya, S. Diagnosis and Management of Duchenne Muscular Dystrophy, Part 1: Diagnosis, and Pharmacological and Psychosocial Management. Lancet Neurol. 2010, 9, 77-93.
64. Long, C.; McAnally, J. R.; Shelton, J. M.; Mireault, A. A.; Bassel-Duby, R.; Olson, E. N. Prevention of Muscular Dystrophy in Mice by Crispr/Cas9-Mediated Editing of Germline DNA. Science 2014, 345, 1184-1188.
65. Ousterout, D. G.; Kabadi, A. M.; Thakore, P. I.; Majoros, W. H.; Reddy, T. E.; Gersbach, C. A. Multiplex Crispr/Cas9-Based Genome Editing for Correction of Dystrophin Mutations That Cause Duchenne Muscular Dystrophy. Nature communications 2015, 6, 6244.
66. Young, C. S.; Mokhonova, E.; Quinonez, M.; Pyle, A. D.; Spencer, M. J. Creation of a Novel Humanized Dystrophic Mouse Model of Duchenne Muscular Dystrophy and Application of a Crispr/Cas9 Gene Editing Therapy. J. Neuromuscul Dis. 2017, 1-7.
67. Young, C. S.; Hicks, M. R.; Ermolova, N. V.; Nakano, H.; Jan, M.; Younesi, S.; Karumbayaram, S.; Kumagai-Cresse, C.; Wang, D.; Zack, J. A. A Single Crispr-Cas9 Deletion Strategy That Targets the Majority of Dmd Patients Restores Dystrophin Function in Hipsc-Derived Muscle Cells. Cell Stem Cell 2016, 18, 533-540.

We claim:

1. A composition for delivering an endonuclease to a cell comprising:
a plurality of self-assembled supramolecular nanoparticles (SMNPs), each of the plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex;
a plurality of terminating components, each having a single terminating binding element that binds to remaining binding regions of one of said plurality of binding components by forming a second inclusion complex;
the endonuclease; and
a nucleotide sequence comprising a recognition sequence specific to the endonuclease,
wherein the plurality of binding components and the plurality of cores self-assemble when brought into contact to form the plurality of self-assembled supramolecular nanoparticles (SMNPs),
wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle,
wherein the endonuclease and the nucleotide sequence form an endonuclease and nucleotide sequence complex, and
wherein the endonuclease and nucleotide sequence complex are encapsulated within each of the plurality of self-assembled supramolecular nanoparticles (SMNPs).

2. The composition of claim 1, wherein the endonuclease is a CRISPR associated protein 9 (Cas9), and
wherein the nucleotide sequence is a single guide RNA (sgRNA).

3. The composition of claim 1, wherein each of the plurality of self-assembled supramolecular nanoparticles (SMNPs) has a diameter of between 40 nanometers and 600 nanometers.

4. The composition of claim 1, wherein the plurality of binding components comprises polythylenimine, poly(L-lysine), or poly(β-amino ester).

5. The composition of claim 1, wherein the plurality of binding regions comprises beta-cyclodextrin, alpha-cyclodextrin, gamma-cyclodextrin, cucurbituril or calixarene.

6. The composition of claim 1, wherein the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

7. The composition of claim 1, wherein the at least one core binding element comprises adamantane, azobenzene, ferrocene or anthracene.

8. The composition of claim 1, wherein the plurality of terminating components comprises polyethylene glycol (PEG) or poly(propylene glycol) (PGG).

9. The composition of claim 1, wherein the single terminating binding element comprises adamantane, azobenzene, ferrocene or anthracene.

10. A system for delivering an endonuclease to a cell comprising:
a plurality of self-assembled supramolecular nanoparticles (SMNPs), each of the plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex;
a plurality of terminating components, each having a single terminating binding element that binds to remaining binding regions of one of said plurality of binding components by forming a second inclusion complex;
the endonuclease; and
a nucleotide sequence comprising a recognition sequence specific to the endonuclease; and
a device for capturing the cell, the device comprising:
a substrate; and
a plurality of nanowires at least one of attached to or integral with a surface of said substrate such that each nanowire of said plurality of nanowires has an unattached end,
wherein the plurality of binding components and the plurality of cores self-assemble when brought into contact to form the plurality of self-assembled supramolecular nanoparticles (SMNPs),
wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle,
wherein the endonuclease and the nucleotide sequence form an endonuclease and nucleotide sequence complex,
wherein the endonuclease and nucleotide sequence complex is encapsulated within each of the plurality of self-assembled supramolecular nanoparticles (SMNPs), and
wherein the plurality of self-assembled supramolecular nanoparticles (SMNPs) are reversibly attached to the plurality of nanowires.

11. The system of claim 10, wherein the endonuclease is a CRISPR associated protein 9 (Cas9), and
wherein the nucleotide sequence is a single guide RNA (sgRNA).

12. The system of claim 10, wherein each of the plurality of self-assembled supramolecular nanoparticles (SMNPs) has a diameter of between 100 nanometers and 150 nanometers.

13. The system of claim 10, wherein the plurality of binding components comprises polythylenimine, poly(L-lysine), or poly(β-amino ester).

14. The system of claim 10, wherein the plurality of binding regions comprises beta-cyclodextrin, alpha-cyclodextrin, gamma-cyclodextrin, cucurbituril or calixarene.

15. The system of claim 10, wherein the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

16. The system of claim 10, wherein the at least one core binding element comprises adamantane, azobenzene, ferrocene or anthracene.

17. The system of claim 10, wherein the plurality of terminating components comprises polyethylene glycol or poly(propylene glycol) (PGG).

18. The system of claim 10, wherein the single terminating binding element comprises adamantane azobenzene, ferrocene or anthracene.

19. The system of claim 10, wherein the plurality of nanowires are grafted with adamantane, adamantane azobenzene, ferrocene, or anthracene.

20. The system of claim 10, wherein the plurality of nanowires has a diameter of between 40 nanometers and 600 nanometers.

21. The system of claim 10, wherein the plurality of nanowires comprises silicon, gold, silver, SiO2, or TiO2.

22. A method for delivering an endonuclease to a cell comprising:
providing a plurality of self-assembled supramolecular nanoparticles (SMNPs); and
contacting the cell with at least one of the plurality of self-assembled supramolecular nanoparticles (SMNPs) such that the at least one of the plurality of self-assembled supramolecular nanoparticles (SMNPs) is taken up by the cell,
wherein the plurality of self-assembled supramolecular nanoparticles (SMNPs) comprise:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex;
a plurality of terminating components, each having a single terminating binding element that binds to remaining binding regions of one of said plurality of binding components by forming a second inclusion complex;
the endonuclease; and
a nucleotide sequence comprising a recognition sequence specific to the endonuclease,
wherein the plurality of binding components and the plurality of cores self-assemble when brought into contact to form the plurality of self-assembled supramolecular nanoparticles (SMNPs),
wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle,
wherein the endonuclease and the nucleotide sequence form an endonuclease and nucleotide sequence complex, and
wherein the endonuclease and nucleotide sequence complex is encapsulated within the plurality of self-assembled supramolecular nanoparticles (SMNPs).

23. The method of claim 22, wherein the endonuclease is a CRISPR associated protein 9 (Cas9), and
wherein the nucleotide sequence is a single guide RNA (sgRNA).

24. The method of claim 22, wherein each of the plurality of self-assembled supramolecular nanoparticles (SMNPs) has a diameter of between 100 nanometers and 150 nanometers.

25. The method of claim 22, wherein the plurality of binding components comprises polythylenimine poly(L-lysine) or poly(β-amino ester),

26. The method of claim 22, wherein the plurality of binding regions comprises beta-cyclodextrin, alpha-cyclodextrin, gamma-cyclodextrin, cucurbituril and calixarene.

27. The method of claim 22, wherein the plurality of cores comprises polyamidoamine dendrimers, poly(prophylenimine) (PPI) dendrimer, triazine dendrimer, carbosilane dendrimer, poly(ether imine) (PETIM) dendrimer or phosphorus dendrimer.

28. The method of claim 22, wherein the at least one core binding element comprises adamantane azobenzene, ferrocene or anthracene.

29. The method of claim 22, wherein the plurality of terminating components comprises polyethylene glycol (PEG) or poly(propylene glycol) (PGG).

30. The method of claim 22, wherein the single terminating binding element comprises adamantane azobenzene, ferrocene or anthracene.

31. The method of claim 22, wherein the plurality of self-assembled supramolecular nanoparticles (SMNPs) are reversibly attached to a plurality of nanowires, and
wherein the plurality of nanowires are at least one of attached to or integral with a surface of a substrate such that each nanowire of said plurality of nanowires has an unattached end.

32. The method of claim 22, wherein the plurality of nanowires are grafted with adamantane azobenzene, ferrocene or anthracene.

33. The method of claim 22, wherein the plurality of nanowires has a diameter of between 40 nanometers and 600 nanometers.

34. The method of claim 22, wherein the plurality of nanowires comprises silicon, gold, silver, SiO2 or TiO2.
